(19) United States
(12) Patent Application Publication
(10) Pub. No.: US 2023/0410959 A1
(43) Pub. Date: Dec. 21, 2023

(54) ELECTRONIC CLINICAL TRIAL PROTOCOL MANAGEMENT SYSTEMS AND RELATED METHODS

(71) Applicant: Flatiron Health, Inc., New York, NY (US)

(72) Inventors: Amit Jindas Shah, Alamo, CA (US); Hugh P. Levaux, Alamo, CA (US)

(73) Assignee: Flatiron Health, Inc., New York, NY (US)

(21) Appl. No.: 18/361,046

(22) Filed: Jul. 28, 2023

Related U.S. Application Data
(62) Division of application No. 17/318,418, filed on May 12, 2021.
Application No. 17/318,418 is a continuation of application No. 15/634,380, filed on Jun. 27, 2017, now abandoned.
(60) Provisional application No. 62/370,228, filed on Aug. 2, 2016.
(60) Provisional application No. 62/360,299, filed on Jul. 8, 2016.
(60) Provisional application No. 62/355,322, filed on Jun. 27, 2016.

(51) Int. Cl. G16H 10/20; G16H 70/20

(52) U.S. Cl. CPC G16H 10/20; G16H 70/20

(57) ABSTRACT

Methods and systems for interacting with and/or managing a clinical trial protocol via an electronic clinical trial protocol management system. The electronic clinical trial protocol management system capable of handling one or more treatment cycles within the clinical trial protocol, and having one or more occurrence programmatic elements and one or more treatment cycle programmatic elements. Each occurrence programmatic element includes a formulaic representation of scheduling of a clinical procedure within one or more treatment cycles. Procedure programmatic elements are related to one or more iterations of a treatment cycle programmatic element. Via such a relationship it is possible to determine a proper clinical procedure applicable to a subject at a position in a clinical trial using an occurrence programmatic element.

### RELATED APPLICATION DATA

This application is a continuation application of U.S. patent application Ser. No. 15/634,380, filed Jun. 27, 2017, entitled “Electronic Clinical Trial Protocol Management Systems and Related Methods,” which is incorporated by reference herein in its entirety.

This application claims the benefit of priority of U.S. Provisional Patent Application Ser. No. 62/355,322, filed Jun. 27, 2016, and titled “Electronic Clinical Trial Protocol Management Systems and Related Methods”; U.S. Provisional Patent Application Ser. No. 62/360,299, filed Jul. 8, 2016, and titled “Electronic Clinical Trial Protocol Management Systems and Related Methods”; and U.S. Provisional Patent Application No. 62/370,228, filed Aug. 2, 2016, and titled “Electronic Clinical Trial Protocol Management Systems and Related Methods”, each of which is incorporated by reference herein in its entirety.

### FIELD OF INVENTION

The present disclosure generally relates to the field of clinical trial management. In particular, the present disclosure is directed to electronic clinical trial protocol management systems and related methods.

### BACKGROUND

Innovation in the clinical trial space continues to occur in the information technology (“IT”) and core science areas. Core science is changing to allow for next generation therapies for extending life and curing cancer (as well as other diseases and ailments). Therapies and clinical trial requirements in many fields (e.g., oncology) are outpacing the abilities of current management systems and related technologies for efficiently running in these clinical trials. Existing IT standards for clinical trial “eProtocol's” (such as CDISC ODM, SD™, and PRM) appear to lack the necessary flexibility for newer clinical trial design.

### SUMMARY OF THE DISCLOSURE

In one exemplary implementation, a computer-implemented method of interacting with a clinical trial protocol via an electronic clinical trial protocol management system, the electronic clinical trial protocol management system being programmed with information describing a clinical trial protocol for a clinical trial having one or more treatment cycles, the electronic clinical trial protocol management system including one or more procedure programmatic elements and one or more treatment cycle programmatic elements, is provided. The method includes receiving via the electronic clinical trial protocol management system an indication of a first position of a subject in the clinical trial, the first position representing at least one of a treatment cycle, an iteration of a treatment cycle, and an interval of an iteration of a treatment cycle for the progression of the subject in the clinical trial; accessing the one or more occurrence programmatic elements in the electronic clinical trial protocol management system, each of the one or more occurrence programmatic elements including information defining a formulaic representation of the scheduling of a clinical procedure within one or more treatment cycles, each formulaic representation including information that relates one or more procedure programmatic elements to one or more iterations of a treatment cycle programmatic element of the one or more treatment cycle programmatic elements, each procedure programmatic element of the one or more procedure programmatic elements including one or more characteristics of a clinical procedure related to the clinical trial; determining one or more select clinical procedures applicable to the subject for the first position using the one or more occurrence programmatic elements; and displaying information related to at least one of the one or more select clinical procedures to a user of the electronic clinical trial protocol management system.

In another exemplary implementation, a machine readable hardware storage medium containing machine executable instructions implementing a method of interacting with a clinical trial protocol via an electronic clinical trial protocol management system, the electronic clinical trial protocol management system being programmed with information describing a clinical trial protocol for a clinical trial having one or more treatment cycles, the electronic clinical trial protocol management system including one or more procedure programmatic elements and one or more treatment cycle programmatic elements, is provided. The method includes a set of instructions for receiving via the electronic clinical trial protocol management system an indication of a first position of a subject in the clinical trial, the first position representing at least one of a treatment cycle, an iteration of a treatment cycle, and an interval of an iteration of a treatment cycle for the progression of the subject in the clinical trial; a set of instructions for accessing the one or more occurrence programmatic elements in the electronic clinical trial protocol management system, each of the one or more occurrence programmatic elements including information defining a formulaic representation of the scheduling of a clinical procedure within one or more treatment cycles, each formulaic representation including information that relates one or more procedure programmatic elements to one or more iterations of a treatment cycle programmatic element of the one or more treatment cycle programmatic elements, each procedure programmatic element of the one or more procedure programmatic elements including one or more characteristics of a clinical procedure related to the clinical trial; a set of instructions for determining one or more select clinical procedures applicable to the subject for the first position using the one or more occurrence programmatic elements; and a set of instructions for displaying information related to at least one of the one or more select clinical procedures to a user of the electronic clinical trial protocol management system.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates one example of a flow chart showing a sample portion of an exemplary clinical trial protocol;

FIG. 2 illustrates another example of a flow chart showing a sample series of iterations of an exemplary treatment cycle;

FIG. 3 illustrates an example of a user interface of an exemplary ECTPM system;

FIG. 4 illustrates one example of a diagrammatic illustration of an exemplary occurrence programmatic element that provides a relationship between a procedure programmatic element and a cycle programmatic element;

FIG. 5 illustrates one example of a diagrammatic illustration of one exemplary formulaic representation;

FIG. 6 illustrates one example of a diagrammatic illustration of another exemplary occurrence programmatic element having a formulaic representation;

FIG. 7 includes one example of a diagrammatic illustration of one exemplary clinical procedure programmatic element that includes an occurrence programmatic element having a formulaic representation;

FIG. 8 includes one example of a diagrammatic illustration of one exemplary treatment cycle programmatic element that includes an occurrence programmatic element having a formulaic representation;

FIG. 9 illustrates one example diagrammatic representation of an ECTPM system;

FIG. 10 illustrates one example of a diagrammatic illustration of an exemplary network disbursed implementation of an example ECTPM system;

FIG. 11 illustrates one exemplary implementation of a method for interacting with a clinical trial protocol via an electronic clinical trial protocol management system;

FIG. 12 illustrates another exemplary implementation of a method for interacting with a clinical trial protocol via an electronic clinical trial protocol management system;

FIG. 13 illustrates yet another exemplary implementation of a method for interacting with a clinical trial protocol via an electronic clinical trial protocol management system;

FIG. 14 illustrates an exemplary association amongst a source document, an electronic copy of a source document, and an electronic signature data element;

FIG. 15 illustrates one exemplary implementation of a combined display of a source document electronic copy and clinical trial data entry;

FIG. 16 illustrates another exemplary implementation of a combined display of a source document electronic copy and clinical trial data entry; and

FIG. 17 shows a diagrammatic representation of one embodiment of a computing device.

### DETAILED DESCRIPTION

Clinical trials exist for testing (e.g., the efficacy) of medical treatments (e.g., pharmaceuticals, medical procedures, etc.) and/or medical devices. Oftentimes clinical trials are regulated by one or more government agencies (e.g., the Food and Drug Administration in the United States). Clinical trials can be managed using electronic systems that aid clinicians in traversing through oftentimes complex clinical trial protocols. A clinical trial protocol can exist in a variety of forms. In one such form, a clinical trial protocol includes a predefined written procedural method in the design and implementation of a clinical trial. A clinical trial protocol can outline the interactions of one or more clinicians with one or more subjects taking part in a clinical trial. Such interactions can include the performance of one or more clinical procedures. An example of a contact point between a subject and a clinician in a clinical trial protocol is a visit. A visit may also be an interaction of a subject and the clinical trial itself that does not necessarily involve contact with a clinician (e.g., a subject taking a pharmaceutical in their home, making a measurement of some clinical characteristic on their own, and/or reporting the same to a trial manager). One or more procedures may be performed during a visit. A more complex event in a clinical trial protocol is a cycle (e.g., a potentially repeating series of interactions between a subject and a trial). Electronically managing cycles has been an ongoing problem for the industry. Existing systems involve lengthy processes of customization of each clinical trial protocol into an electronically codified form that is not easily modified for changes and/or variations in that protocol or easily ported to a wholly different protocol. This is partly due to the requirement of some known systems for encoding in long form each visit and each iteration of a repeating cycle into the programmatic code of a system. For example, for a given portion of a clinical trial protocol, each visit and each interval of every iteration of a cycle must be coded in order into the computerized system. In such a system, the designer must have a set number of iterations of a cycle to program into the computerized system. Thus, existing systems have problems as the complexity of a protocol increases and the number of iterations of a cycle become more indefinite (e.g., being based on the interaction of the trial with the subject and/or the responses of the subject to treatment with a drug or device). This problem is made greater by the regulatory requirements on validation of changes to electronic codifications of clinical trial protocols.

For example, when a pharmaceutical company wants to initiate the development of an electronic system for management and/or interaction with a clinical trial protocol that it has developed, it may need to enlist a software programmer to manually code each visit and each iteration of a cycle (including each visit or other procedural interaction with a subject in each iteration) in long form code into the electronic management system. In this way, it would need to designate a discrete number of iterations for each treatment cycle into the code in the order in which they would occur in the treatment protocol. One problem with this approach is the reality in many implementations of clinical trial protocol, the designer does not know the exact number of iterations of a cycle that will be needed to achieve a certain result (e.g., a certain reaction by a subject to taking a treatment of a pharmaceutical) and, thus, does not know the number of iterations to long-form encode into the electronic management system. In addition to requiring major time and resources to implement, changes to the long form encoding can result in a need for revalidation under regulatory processes. Thus, adding more time to the process.

The current disclosure includes various computerized methods and systems for interaction with a clinical trial protocol. For example, improvements to the systems and methods related to the specialized computer systems utilized in the clinical trial space for electronically modeling and interacting with a clinical trial protocol are provided. Examples of an interaction with a clinical trial protocol include, but are not limited to, electronically modeling a clinical trial protocol, electronically collecting information and/or data related to a clinical procedure of a clinical trial protocol, electronically storing an electronic copy of a clinical trial source document, visually displaying information of a clinical trial protocol (e.g., information related to a clinical procedure, a visit, a cycle, an iteration of a cycle, an interval of a protocol, an interval of a cycle), interacting with a medical record of a subject, theoretically modeling an instantiation of one or more portions of a clinical trial protocol for analysis (and possible output) of data related to the same, and any combinations thereof. Such improvements and interactions may be included in an exemplary electronic clinical trial protocol management system (ECTPM system) of the current disclosure. In an example, an electronic clinical trial protocol management system may include functionality of an electronic data collection (EDC) system, a CTMS system, and/or an electronic medical records (EMR) system.

A clinical trial protocol can include various components in describing how a clinical procedure is to be implemented. Examples of such components include, but are not limited to, a clinical procedure, a visit, a treatment cycle, an interval at which actions (e.g., a procedure, a visit, a cycle iteration, etc.) occur, and any combinations thereof. An ECTPM system may model one or more of such components as a programmatic element as is described further below.

A clinical procedure includes one or more tasks performed in a contact point between a subject and a clinical trial. There are various clinical procedures that are included in a clinical trial. Any known clinical procedure may be performed in a clinical trial depending on the clinical trial (e.g., its design and/or a desired measurement for the trial, etc.). Examples of a subject include, but are not limited to, a human subject, an animal subject, a theoretical subject (e.g., a data instantiation of a subject within a modeling of a clinical trial protocol wherein the theoretical subject is not a real world subject, a theoretical data instantiation of a real world subject in a modeled clinical trial protocol), and any combinations thereof. Examples of a clinical procedure include, but are not limited to, a measurement of a subject characteristic (e.g., a vital sign, a blood pressure, a temperature, a body weight, etc.), an imaging of a subject (e.g., an x-ray, an x-ray computed tomography (x-ray CT), a computerized axial tomography scan (CAT scan), a magnetic resonance imaging (MRI)), another instrument reading of a subject characteristic (e.g., an electrocardiogram (ECG/EKG), electroretinogram (ERG), an electrogastrogram (EGG), electrogastroenterogram, electroencephalogram (EEG)), taking a medical history of a subject, having a subject rest without treatment for one or more intervals, and any combinations thereof.

A visit is an interaction point of a subject with a clinical trial. A visit may involve one or more clinical procedures. A clinical trial protocol typically schedules a particular visit to occur at a particular interval in the clinical trial. In one example, a visit occurs as a physical contact point between a subject and one or more clinicians involved in a clinical trial. In another example, a visit may involve a subject performing one or more procedures independent of a physical contact with a clinician or a clinic (e.g., performing a procedure at home, collecting clinical data using a user-friendly portable device, etc.). Examples of an interval for use in a clinical trial include, but are not limited to, a second, a minute, a day, a week, a biweek, a month, a year, and any combinations thereof. In one example, an interval used to space events within a clinical trial is a day. A clinical trial may include some events spaced at one interval unit and other events spaced apart at a second interval unit (e.g., some visits spaced at intervals of days and other visits spaced at intervals of weeks). Multiple clinical trial “visits” may occur in one physical visit of a subject to a clinician (e.g., the “visits” may be spaced at intervals of minutes such that the subject may only have a single physical visit in a given day to a clinic to undergo the procedures associated with the multiple visits).

A treatment cycle is a course of treatment (e.g., a set of visits and/or procedures) that is repeated on a regular schedule. In some examples, a treatment cycle includes a period of treatment (e.g., of a pharmaceutical, of a medical device) and a period of rest. In such an example, the treatment/rest periods can repeat as needed. A clinical trial protocol treatment cycle may include information describing visits and/or procedures (including possible subject rest periods) that will occur and the timing of each at intervals within the treatment cycle. For example, a subject may be administered a drug under trial on a first interval of a cycle with different measurements and reading of the subject occurring at later intervals in the cycle. These visits and/or procedures may repeat for one or more desired reasons of the protocol design. For example, if certain criteria are met (and/or are not met) regarding the subject in the treatment (e.g., the subject has characteristics of improvement from a drug, the subject has not met a threshold of improvement from a drug treatment, etc.) a cycle may repeat. Each instance of the cycle (i.e., each repetition) may be referred to as an iteration of that cycle. In common discussion in the clinical trial field, the term “cycle” may be used to refer to each iteration of a cycle (e.g., “the subject is in cycle number 5 of the xyz drug administration”). In this disclosure, the term “iteration” will be used for each instance of a cycle (including the first instance of a cycle). Similar to the protocol as a whole, events occur in a treatment cycle at intervals. In this disclosure, the intervals of a cycle may be referred to as “cycle intervals.” A cycle may have any length (e.g., number of intervals). Also, a cycle may have visits and/or procedures occurring at intervals at any time during a cycle iteration. In some examples, different iterations of a cycle may have different numbers of intervals and may have different procedures occurring on different intervals of that iteration. The length of a treatment phase and a rest phase, when utilized, may vary from one iteration of a cycle to another. Thus, cycle implementation in a clinical trial protocol can vary in complexity and sometimes has a great level of complexity.

As discussed above, the number of iterations of a cycle for a given subject progressing through a treatment cycle may not be known at the time of designing a protocol (e.g., not knowing how a given subject will react to a treatment and/or how long it will take for the subject to reach a desired point in the treatment of the cycle).

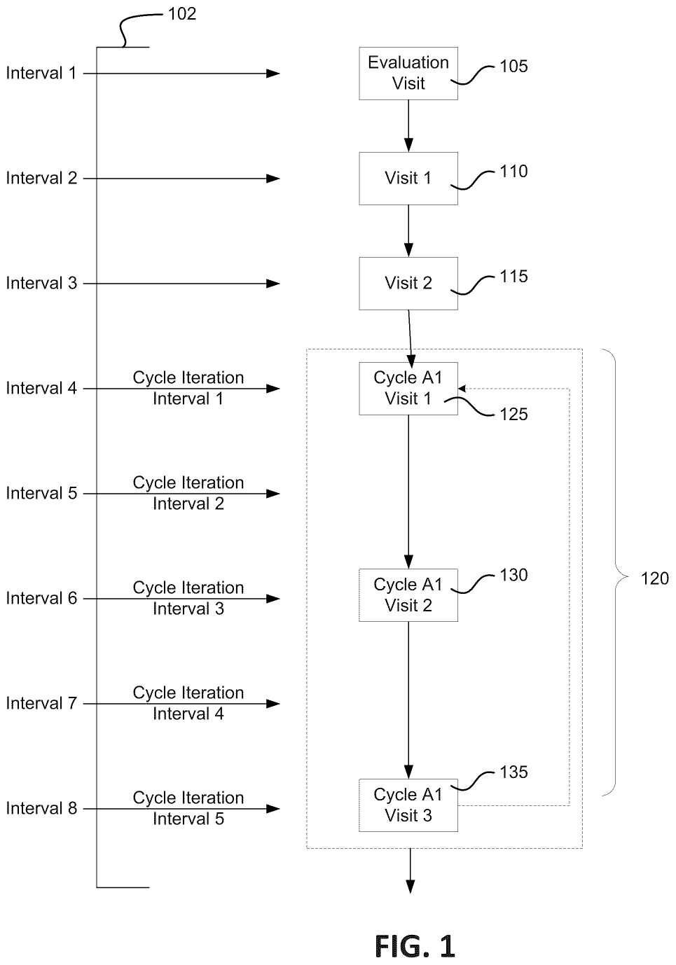

FIG. 1 illustrates one example of a flow chart showing a portion of an exemplary clinical trial protocol. On the left of the illustration is shown a timeline 102 that is divided into intervals. Interval 1 through Interval 8 are shown for this portion of the clinical trial protocol. If a given instantiation of this protocol does not end on a visit occurring at Interval 8, additional intervals will occur after Interval 8. Timeline 102 progresses in time downward from the top of the timeline to the bottom. At the beginning of the trial protocol is an evaluation visit 105 that occurs at Interval 1. A visit 110 occurs at the next interval, Interval 2, and a visit 115 occurs at Interval 3. After visit 115, a subject enters into a cycle 120 that starts at Interval 4. Cycle 120 is shown having an identifier of “A1.” Cycle 120 includes three visits (visits 135, 140, 145) that may iterate n number of times depending on the circumstances of a given subject and the interaction of that subject with the trial protocol (e.g., interaction with a pharmaceutical under trial). Visit 125 occurs at Interval 4 of the trial. Interval 4 corresponds with the first interval (“Cycle Interval 1”) for the first iteration of cycle 120. Visit 125 occurs at Cycle Interval 1. Visit 130 occurs at Cycle Interval 3. There is no visit in this example protocol at Cycle Interval 2 or Cycle Interval 4. These intervals are skipped. Visit 135 occurs at Cycle Interval 5. Depending on the conditions of the protocol and the subject in a particular instantiation, cycle 120 may repeat (e.g., the second iteration of cycle 120 starting at Interval 9 of the protocol). If cycle 120 does not repeat after a given iteration for a subject, the protocol will proceed to the next stage of the protocol (not shown) (e.g., another cycle, a general visit, an exit of the subject from the trial, an end to the trial for the subject, a branching of the trial protocol, etc.).

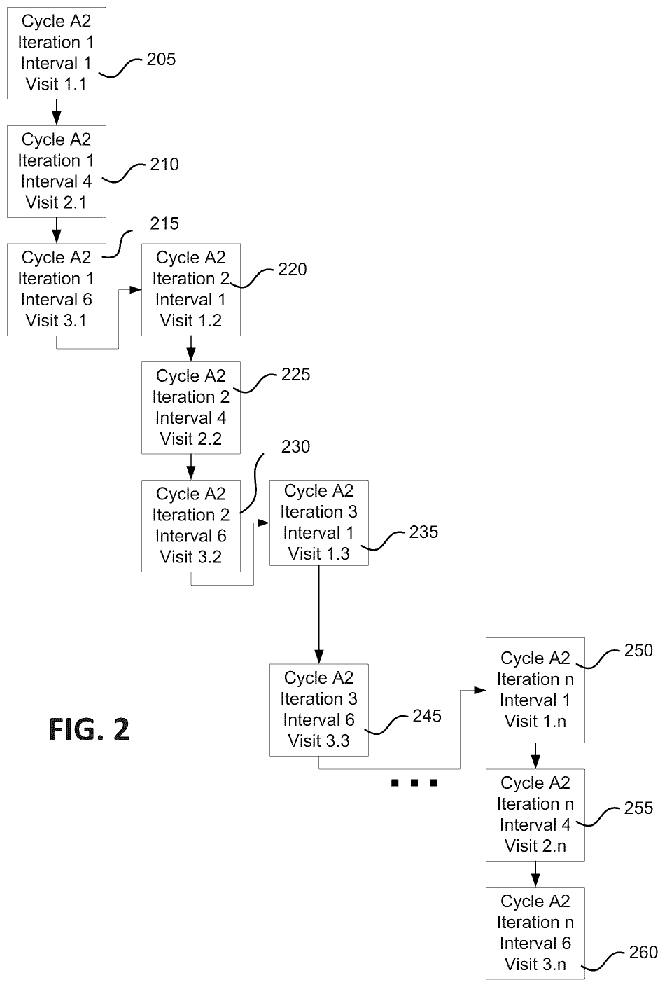

FIG. 2 illustrates an example of a flow chart showing a series of iterations of an exemplary treatment cycle “A2.” Iteration 1 of cycle A2 includes visits 205, 210, and 215 (which correspond to visit identifications 1, 2, and 3, respectively). Visit 205 occurs on cycle interval 1, visit 210 occurs on cycle interval 4, and visit 215 occurs on cycle interval 6 (cycle intervals 2, 3, and 5 have no visits and are skipped). After visit 215, cycle A2 starts a second iteration (cycle iteration 2) that includes visits 220, 225, and 230. Visits 220, 225, and 230 are iterations of visit identifications 1, 2, and 3, respectively). In iteration 2, visits 220, 225, and 230 occur on cycle intervals 1, 4, and 6, respectively with intervals 2, 3, and 5 having no visits. After visit 230, cycle A2 starts a third iteration (cycle iteration 3) that includes visits 235 and 245. Visit 235 occurs on cycle interval 1 and visit 245 occurs on cycle interval 6. It is noted that for this exemplary protocol, iteration 3 of cycle A2 calls for no visit on cycle interval 4 as in other intervals. In iteration 3 there is no visit on interval 2, 3, 4, or 5. The ellipse between visit 245 and a visit 250 of cycle iteration n are intended to show that the number of iterations of cycle A2 is a variable number. Iteration n of cycle A2 includes visit 250 and visits 255 and 260. Visits 250, 255, and 260 occur on cycle intervals 1, 4, and 6, respectively (with no visits on cycle iterations 2, 3, and 5). FIG. 2. Illustrates a repeating pattern of cycle iterations.

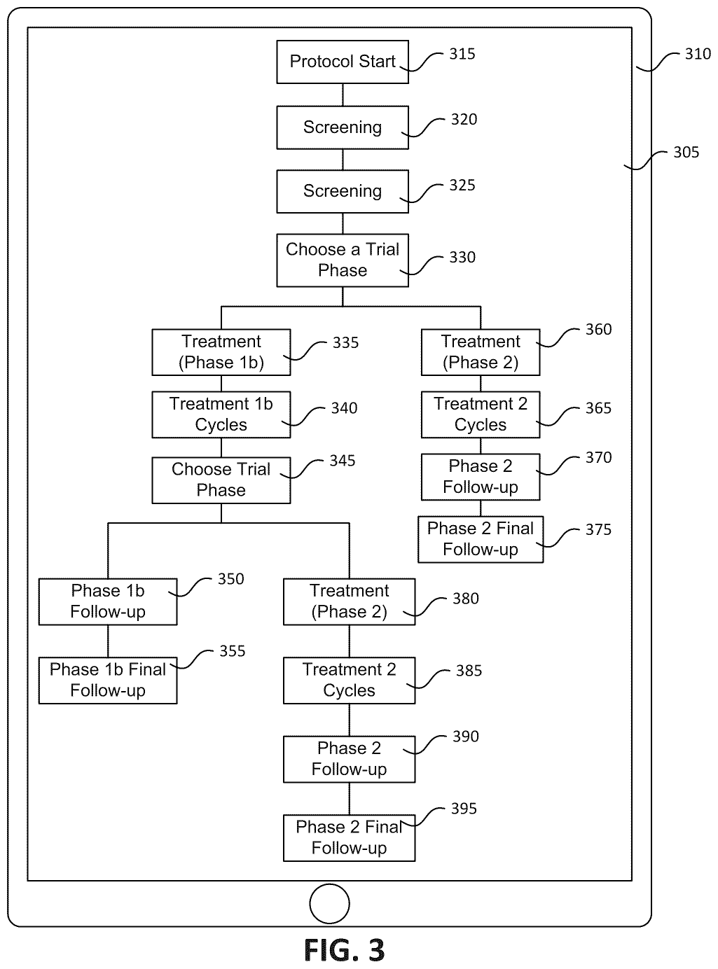

FIG. 3 illustrates an example of a user interface of an exemplary ECTPM system. The user interface is shown as a display 305 on a computerized tablet device 310. The display illustrates boxes in a flow chart that branches. Each box represents a visit or a treatment cycle within a protocol example. Box 315 represents a clinical trial protocol start. Boxes 320 and 325 represent visits for screening. Box 330 represents a decision for placing a subject into one of multiple phases (in this example Phase 1b and Phase 2). Phase 1b is represented by box 335 (a treatment visit), box 340 (a treatment cycle for Phase 1b), and box 345 (a choice for different phases: complete Phase 1b or start Phase 2). Completion of Phase 1b is represented by box 350 (a follow-up visit) and box 355 (a final follow-up visit on Phase 1b). Phase 2 is represented at the first decision box 330 by box 360 (a treatment visit), box 365 (a treatment cycle for Phase 2), box 370 (a follow-up visit), and box 375 (a final follow-up visit). Phase 2 as a branch of Phase 1b is represented by box 380 (a treatment visit), box 385 (a treatment cycle for Phase 2), box 390 (a follow-up visit), and box 395 (a final follow-up visit).

An electronic clinical trial protocol management system can be specially programmed to implement any combination of the functionalities described herein. An ECTPM system may include a set of interrelated electronic programmatic elements for specifically interacting with the ECTPM system to achieve such a functionality. In one example, a set of interrelated electronic programmatic elements is included for specifically interacting with the ECTPM system to model a flexible number of treatment cycle iterations for a given subject progressing through a clinical trial that is represented by the ECTPM system allowing for one or more of the interactions with the ECTPM system discussed herein. A programmatic element is a selection of computerized code (e.g., one or more related elements of a tabular formatted computer system, one or more objects in an object-oriented computer system, etc.) implementable via a specially programmed computer machine to perform a specified functionality within an ECTPM system. Examples of a programmatic element in an ECTPM system of the current disclosure include, but are not limited to, an occurrence programmatic element, a clinical procedure programmatic element (possibly referred to herein as a procedure programmatic element), a treatment cycle programmatic element (possibly referred to herein as a cycle programmatic element), a visit programmatic element, and any combinations thereof.

In one exemplary implementation, an electronic clinical trial protocol management system includes an occurrence programmatic element. An occurrence programmatic element includes information and/or programming for relating one or more procedure programmatic elements to one or more iterations of a treatment cycle programmatic element. A procedure programmatic element includes information and/or programming representing one or more characteristics of a clinical procedure of a clinical trial represented by a clinical trial protocol. Example information for representing a clinical procedure includes, but is not limited to, a procedure identification information, a name/title information, notice (or other descriptive information), process step information, data information for data required to implement a procedure, data information for data input corresponding to a result of a procedure (e.g., a pointer to a clinical trial source document or electronic copy thereof related to implementation of a procedure, actual data resulting from a procedure, such as a blood pressure result from a subject), guidance information (e.g., information to guide a clinician in the implementation of a procedure, and any combinations thereof.

A treatment cycle programmatic element includes information and/or programming defining a treatment cycle of a clinical trial represented by a clinical trial protocol. Example information for representing a clinical procedure includes, but is not limited to, a cycle identification information, a name/title information, notice (or other descriptive information), interval unit information for the units of intervals applicable to a cycle (e.g., days, minutes, weeks, etc.), information representing a minimum number of intervals for a cycle, information representing a maximum number of intervals for a cycle, information representing a minimum number of cycle iterations required of a subject progressing through a cycle, information representing a maximum number of cycle iterations required of a subject progressing through a cycle, data information for data required to implement a cycle, data information for data input corresponding to an interaction with a cycle (e.g., a pointer to a clinical trial source document or electronic copy thereof related to a cycle, actual data input related to a cycle), guidance information (e.g., information to guide a clinician in the implementation of a cycle), scheduling/occurrence information for any of the other information, interval window information (see further description below), and any combinations thereof. A cycle programmatic element may include information related to a clinical trial visit programmatic element. A visit programmatic element includes information and/or programming defining a visit of a clinical trial represented by a clinical trial protocol.

A programmatic element may be encoded using a computer coding format that is compatible with the functionality of the given programmatic element. Examples of a computer coding format include, but are not limited to, a tabular data format (e.g., a comma-separated value format (CSV), an SAS XPORT (XPT) format, an Microsoft Excel (XLS) or other spreadsheet format), a Microsoft Access format, a structured query language (SQL) format), an object-oriented design data format (e.g., an extensible markup language (XML) format, a JavaScript Object Notation (JSON) format, a binary JSON (BSON) format), a protocol buffers format, a message pack format, a human-readable data serialization language (e.g., YAML) format, a standard generalized markup language (SGML) format, a hypertext markup language (HTML) format, and any combinations thereof. In one example, a JSON data format is utilized for programmatic elements in an ECTPM system.

A relating of one or more procedure programmatic elements to one or more iterations of a treatment cycle programmatic element can include an occurrence formulaic representation (also referred to herein in short as a formulaic representation). In one example, a formulaic representation may utilize a formula for making a relationship such that the connection of a procedure programmatic element to an iteration of a treatment cycle programmatic element is done in a manner that allows creation during modeling and interaction of a flexible number of iterations of a treatment cycle programmatic element. In one such example, a formulaic representation includes information from a clinical trial protocol design that allows for the creation by the ECTPM system of a number of iterations of a treatment cycle represented by the treatment cycle programmatic element to occur in a number of iterations that may not be known at the time that the treatment cycle programmatic element and/or the procedure programmatic element is coded into the ECTPM system. A formulaic representation may include one or more specific scheduling characteristic information elements. including information defining at which iteration of a cycle (and optionally, which interval within an iteration of a cycle) a procedure will occur. Examples of a scheduling characteristic information elements include, but are not limited to, an indicator of interval for implementation of a clinical procedure, an indicator of a starting cycle iteration for implementation of a clinical procedure, an indicator of an ending cycle iteration for implementation of a clinical procedure, and an indicator of cycle iterations to skip implementation of a clinical procedure, and any combinations thereof.

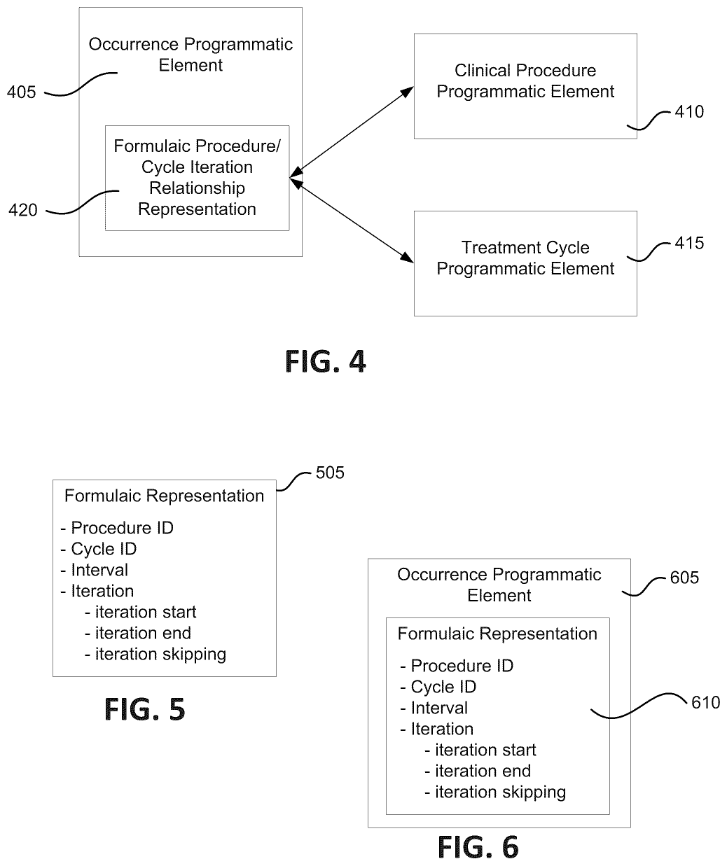

FIG. 4 illustrates one example of a diagrammatic illustration of an exemplary occurrence programmatic element 405 that provides a relationship between a procedure programmatic element 410 and a cycle programmatic element 415. Occurrence programmatic element 405 includes a formulaic procedure/cycle iteration relationship representation 420. One exemplary benefit of organizing programmatic elements in such a fashion and including such a formulaic representation is an improvement to the particular iteration problems of the prior art ECTPM-type systems that utilize long form encoding. Such a particular type of shorter-form encoding allows for variations in the number of iterations in a cycle for a given subject as they progress through a clinical trial. In one example, a formulaic representation (e.g., representation 420, representation 505 below) includes information and/or programmatic coding that allows an ECTPM system of the current disclosure to model a variable number of treatment cycle iterations for a subject in a clinical trial of the ECTPM system. In one such example, a formulaic representation includes information and/or programmatic coding that allows an ECTPM system to model a number of treatment cycle iterations for a subject in a clinical trial of the ECTPM system without modification to a procedure programmatic element coding or a treatment cycle programmatic element coding of the ECTPM, wherein the number of treatment cycle iterations was not known at the time that the procedure programmatic element or the treatment cycle programmatic element were coded for the ECTPM system.

FIG. 5 illustrates one example of a diagrammatic illustration of one exemplary formulaic representation 505 (e.g., for use such as representation 420). Formulaic representation 505 includes the following scheduling characteristic information elements: one or more procedure identification elements (“Procedure ID”), one or more cycle identification elements (“Cycle ID”), one or more interval identification elements (“Interval”), and one or more cycle iteration elements (“Iteration”). A procedure identification element of a formulaic representation is a programmatic sub-element that identifies a procedure programmatic element of the ECTPM system that represents a clinical procedure that is to be performed related to a subject in a progression of a clinical trial. Each procedure identification element includes one or more cycle identification elements (a programmatic sub-element that identifies a cycle programmatic element of the ECTPM system that represents a treatment cycle of the protocol in which the corresponding clinical procedure will occur. One or more cycle interval identification elements is included and each has information identifying an interval of the corresponding treatment cycle at which the corresponding procedure will occur. A cycle iteration element includes programmatic information describing which cycle iterations will have the corresponding procedure occurring at the listed interval. In the example of representation 505, the cycle iteration element includes programmatic information describing a starting cycle iteration for implementation of a clinical procedure (“iteration start”), an indicator of an ending cycle iteration for implementation of a clinical procedure (“iteration end”), and an indicator of cycle iterations to skip implementation of a clinical procedure (“iteration skipping”). An iteration start, iteration end, and/or iteration skipping programmatic sub-element may be omitted if such information is not needed (e.g., if a procedure occurs at every iteration of a cycle, there would be no need for a iteration skipping information or an iteration end information). ID references in a formulaic representation for a procedure and/or a cycle may refer to a corresponding procedure programmatic element and/or a cycle programmatic element, respectively.

In one exemplary implementation of formulaic representation 505, a clinical trial protocol may call for an EKG procedure to occur in a treatment cycle “A3” at a visit on the 5^{th} day (cycle interval=day) of the cycle. However, the EKG procedure in this example is only required on the 3^{rd} cycle iteration and then only every 2 cycle iterations thereafter (ending on the 10^{th} iteration). In the corresponding ECTPM system, a procedure programmatic element exists with an ID of “EKG” corresponding to the EKG clinical procedure of the trial. The ECTPM system also includes a treatment cycle programmatic element with an ID of “A3” to correspond to the treatment cycle “A3” of the clinical trial. In such an example, a formulaic representation of an occurrence programmatic element may appear as follows:
Procedure ID: “EKG”
Cycle ID: “A3”
Interval: 5
Iteration:
   iteration start: 3
   iteration end: 10
   iteration skipping: 2

A formulaic representation, such as representation 505, may optionally include an interval window programmatic subelement. In some examples of a clinical trial protocol design, it is acceptable for a procedure and/or visit that is scheduled to occur at an interval of a trial and/or an interval of a cycle to occur at one or more intervals before the scheduled interval and/or one or more intervals after the scheduled interval. For example, if an EKG procedure is scheduled to occur on interval 5 of cycle A3, it may be acceptable that the EKG occur as early as interval 3 and as late as interval 6 of an iteration of cycle A3. An interval window programmatic element is a formulaic representation of a period of time (e.g., a number of intervals) before a scheduled interval and/or a period of time (e.g., a number of intervals) after a scheduled interval at which a subject may have an interaction with the trial (e.g., a visit and/or procedure) that is compliant with the interval at which the interaction is scheduled to occur. An interval window programmatic element may also be part of a cycle programmatic element (see an example below).

FIG. 6 illustrates one example of a diagrammatic illustration of another exemplary occurrence programmatic element 605 having a formulaic representation 610. Formulaic representation 610 includes the following scheduling characteristic information elements: one or more procedure identification elements (“Procedure ID”), one or more cycle identification elements (“Cycle ID”), one or more interval identification elements (“Interval”), and one or more cycle iteration elements (“Iteration”). These scheduling characteristic information elements have similar aspects, features, and functionalities as discussed above with respect to other formulaic representations.

An occurrence programmatic element, such as element 405, 605, may occur in an ECTPM as an element that is programmatically separate from corresponding procedure programmatic elements and cycle programmatic elements (e.g., is a separate object code object, is a separate tabular code object). In another example, an occurrence programmatic element may be part of a procedure programmatic element. In yet another example, an occurrence programmatic element may be part of a cycle programmatic element. It is possible for a given ECTPM system implementation to have occurrence programmatic elements of different code location types.

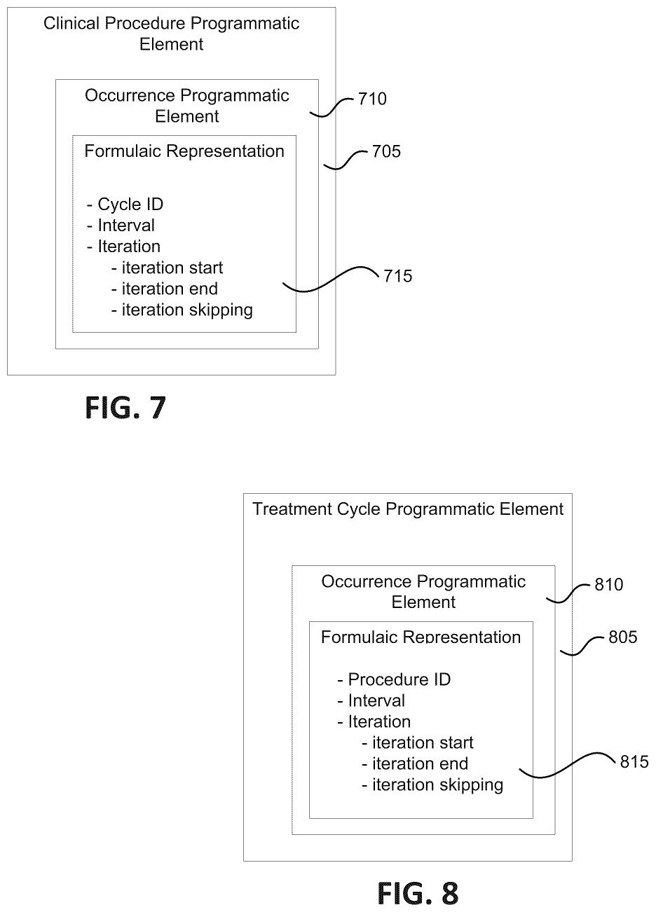

FIG. 7 includes one example of a diagrammatic illustration of one exemplary clinical procedure programmatic element 705 that includes an occurrence programmatic element 710 having a formulaic representation 715. Formulaic representation 715 includes the following scheduling characteristic information elements: one or more cycle identification elements (“Cycle ID”), one or more interval identification elements (“Interval”), and one or more cycle iteration elements (“Iteration”). These scheduling characteristic information elements have similar aspects, features, and functionalities as discussed above with respect to other formulaic representations. It is noted here that in this example, a procedure identification element (“Procedure ID”) is not required as the formulaic representation appears as part of the corresponding procedure programmatic element.

The following computing coding illustrates another example of a procedure programmatic element (“id”: 11), written in a JavaScript Object Notation (JSON) format, that includes an occurrence programmatic element positioned within the object code of the procedure programmatic element. In the example below for procedure programmatic element (“id”: 11), the clinical procedure identification information is “id”: 11 and the title information for the procedure is represented as “name”; “ABC 103 (1.5-4 hour infusion)”. The procedure programmatic element includes within its structure an occurrence programmatic element (“occurrences”) that includes an indication of which visit (represented by the relationship to multiple visit programmatic elements) at which the procedure will occur in the clinical trial (“visits”:). Additionally, the occurrence programmatic element includes a relationship to a treatment cycle programmatic element (cycle “id”: 1). The corresponding ECTPM system would include a corresponding treatment cycle programmatic element that would have the information for defining that treatment cycle (see above for examples). Although in this example, only one cycle programmatic element is referenced (e.g., due to the procedure ID 11 only occurring in that cycle), additional cycle programmatic elements could be referenced in the “cycles”: portion of the occurrence programmatic element. The “cycles”: portion of the occurrence programmatic element includes a formulaic representation that includes an indication at which interval of the cycle the procedure occurs (interval 1) and a “schedule” sub-element that defines the starting iteration (1) of the cycle at which the procedure occurs and an indication of skipping (“every”) of iterations, which in this example has no skipping in that the procedure will be at every 1 iteration.

```
{
  “id”: 11,
  “name”: “ABC 103 (1.5 − 4 hr Infusion)”,
  “occurrences”: {
    “visits”: [
      3,
      4,
      5,
      6,
      7,
      8,
      9,
      10,
      11,
      12,
      13,
      14,
      15
    ],
    “cycles”: [
      {
        “id”: 1,
        “intervals”: [ 1 ],
        “schedule”: [
          {
            “start”: 1,
            “every”: 1
          }
        ]
      }
    ]
  }
},
```

FIG. 8 includes one example of a diagrammatic illustration of one exemplary treatment cycle programmatic element 805 that includes an occurrence programmatic element 810 having a formulaic representation 815. Formulaic representation 815 includes the following scheduling characteristic information elements: one or more procedure identification elements (“Procedure ID”), one or more interval identification elements (“Interval”), and one or more cycle iteration elements (“Iteration”). These scheduling characteristic information elements have similar aspects, features, and functionalities as discussed above with respect to other formulaic representations. It is noted here that a cycle identification elements (“Cycle ID”) is not required as the formulaic representation appears within a cycle programmatic element.

The following computing coding illustrates another example of a treatment cycle programmatic element, having cycle identification (“id”: 1) encoded in a JSON format. In this example, the cycle programmatic element includes information for describing the treatment cycle: a “name”, a definition of the interval unit (“interval units”: “day”) with an interval length of a day, a minimum number of iterations of the cycle (“min”: 1), a notice sub-element utilized for informational display to a user (“notice”:), a length schedule that defines the number of intervals in each iteration of the cycle (“length_schedule”:), an interval window definition which defines the number of intervals before a scheduled interval and a number of intervals after a scheduled interval during which a procedure scheduled for that scheduled interval can permissibly occur (“interval_windows”:). In this example, the relationship by an occurrence programmatic element (“procedures”:) is done by the occurrence programmatic element set forth within the cycle programmatic element. In this approach, the procedures that apply to the cycle represented by the cycle programmatic element. For purposes of keeping the listed encoding short, the occurrence programmatic element is shown with only two procedures listed (e.g., “procedure_id”: 1 and “procedure_id”: 4). It is noted that any number of procedures could be related via this occurrence programmatic element by adding additional terms to describe such a procedure relation. Procedure ID 1 sub-element includes the “cycles” sub-element that describes the interval at which procedure ID 1 will occur in the cycle (“intervals”: [1]). It is noted that more than one interval can be listed such an interval identifier (e.g., by listing a series of interval numbers). The “schedule”: sub-element includes a formulaic description of the starting iteration (“start”: 1) and the iteration skipping information (“every”: 1) that describes that procedure ID 1 will occur starting in the first iteration of the cycle and every cycle iteration thereafter on the first day of the cycle (interval 1). Procedure ID 4 sub-element includes the “cycles” sub-element that describes the interval at which procedure ID 4 will occur in the cycle (“intervals”: [3]). It is noted that more than one interval can be listed such an interval identifier (e.g., by listing a series of interval numbers). The “schedule”: sub-element includes a formulaic description of the starting iteration (“start”: 1), ending iteration (“end”: 6), and the iteration skipping information (“every”: 1) that describes that procedure ID 4 will occur starting in the first iteration of the cycle and every other cycle iteration thereafter (i.e., skipping one iteration in between) on the third day of the cycle (interval 3) until the sixth iteration at which it will no longer repeat.

```
cycle: [
  {
    ″id″: 1,
    ″name″: ″albortonib″,
    ″interval_units″: ″day″,
    ″min″: 1,
    ″notice″: [
      {
        ″criteria″: [
          {
            ″text″: ″test me cycle″
          }
        ],
        ″schedule″: [
          {
            ″interval″: 7,
            ″schedule″: [
              {
                ″start″: 1,
                ″every″: 1
              }
            ]
          }
        ]
      }
    ],
    ″length_schedule″: [
      {
        ″length″: {
          ″treatment″: 14,
          ″rest″: 7
        },
        ″schedule″: [
          {
            ″start″: 1,
            ″every″: 1,
            ″end″: 9
          }
        ]
      },
      {
        ″length″: {
          ″treatment″: 14,
          ″rest″: 28
        },
        ″schedule″: [
          {
            ″start″: 10,
            ″every″: 1
          }
        ]
      }
    ],
    ″interval_windows″: [
      {
        ″interval″: 7,
        ″window″: {
          ″before″: 1,
          ″after″: 1
        },
        ″schedule″: [
          {
            ″start″: 1,
            ″every″: 1
          }
        ]
      },
      {
        ″interval″: 14,
        ″window″: {
          ″before″: 1,
          ″after″: 1
        },
        ″schedule″: [
          {
            ″start″: 1,
            ″every″: 1,
            ″end″: 9
          }
        ]
      },
      {
        ″interval″: 14,
        ″window″: {
          ″before″: 2,
          ″after″: 2
        },
        ″schedule″: [
          {
            ″start″: 10,
            ″every″: 1
          }
        ]
      }
    ],
    ″procedures″: [
      {
        ″procedure_id″: 1,
        ″cycles″: [
          {
            ″intervals″: [ 1 ],
            ″schedule″: [
              {
                ″start″: 1,
                ″every″: 1
              }
            ]
          }
        ]
      },
      {
        ″procedure_id″: 4,
        ″cycles″: [
          {
            ″intervals″: [ 3 ],
            ″schedule″: [
              {
                ″start″: 1,
                “end”: 6,
                ″every″: 2
              }
            ]
          }
        ]
      }
    ]
  }
]
```

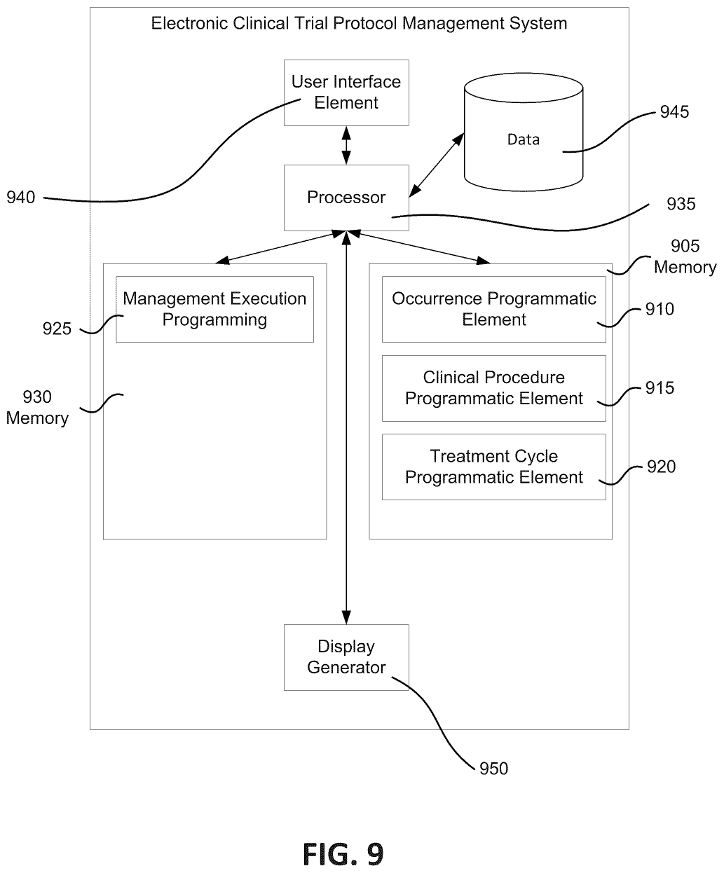

FIG. 9 illustrates one example diagrammatic representation of an ECTPM system 900 including one or more of the aspects, features, implementations, etc. of the current disclosure for improved operation of an electronic clinical trial protocol. System 900 includes a memory 905 including a set of occurrence programmatic elements 910, a set of clinical procedure programmatic elements 915, and a set of treatment cycle programmatic elements 920. Example memories are discussed further below. A programmatic element of the current disclosure can be readily modified by a user of an ECTPM system, such as system 900, to make minor modifications to such programmatic elements in some cases without a need for extensive long form encoding of sequential steps of a protocol and/or without a need for revalidation of program execution coding under a regulatory process. For example, system 900 also include management execution programming 925 for including software and other instructions for general operation of the ECTPM system 900 with reference to programmatic elements, such as occurrence programmatic element 910, procedure programmatic element 915, and/or treatment cycle programmatic element 920 (e.g., to formulaically relate an iteration of a cycle with a procedure to be implemented at an interval of that iteration). In one such example, ECTPM system 900 can generate a flexible number of iterations of a cycle based on occurrence programmatic element 910 (e.g., and possibly allow changes to a scheduling characteristic information elements thereof to modify characteristics of the protocol, such as which cycle interval a procedure occurs and/or at which iteration of a cycle).

Management execution programming 925 is shown stored at memory 930. Memory 905 and 930 are shown as separate components. It is understood that any one or more components of an ECTPM system can be combined (even if shown separately), separated into two or more like components (even if shown as one), distributed across one or more computing devices (e.g., upon one or more server computers and one or more client computers), geographically distributed (e.g., utilizing one or more networks), and any combinations thereof. Memories 905, 930 are shown connected to a processor 935. Example processors are discussed further below. Processor 935 accesses programmatic elements 910, 915, 920 and execution programming 925 to implement details, concepts, aspects, features, characteristics, examples, and/or alternatives of the current disclosure (e.g., relating an iteration of a cycle to a procedure using an occurrence programmatic element). System 900 also includes a user interface element 940 connected to processor 935 for allowing a user to interact with system 900 and one or more of its components. Various user interfaces are discussed below and include, but are not limited to, a scanner, a display element, a data input element (e.g., a keyboard, a pointer device, a touch screen, etc.), a network connection (e.g., to associate a source document with system 900, etc.), and any combinations thereof.

System 900 includes a display generator 950. Examples of a display generator are discussed further below and include, but are not limited to, a display device, a graphical interface display hardware, software and/or other machine/computer executable instructions for producing a displayable image, and any combinations thereof. Display generator 950 is connected with processor 935 to allow system 900 to produce a displayable image of one or more information related to ECTPM system 900 and/or its operation and/or a clinical trial protocol modeled thereon.

System 900 includes a data storage 945 for storing data related to the operation of system 900. Data storage 945 may include a memory. Example data that can be stored in relation to system 900 and/or data storage 945 includes, but is not limited to, data related to interaction of a subject with a clinical trial of system 900, data representing an instantiation of a clinical trial and/or any one or more of its components on system 900 (e.g., data representing a cycle iteration generated by system 900), an electronic copy of a source document, a clinical trial source document, a an electronic signature data element, data linking any data element and/or programmatic element of system 900 to another, and any combinations thereof.

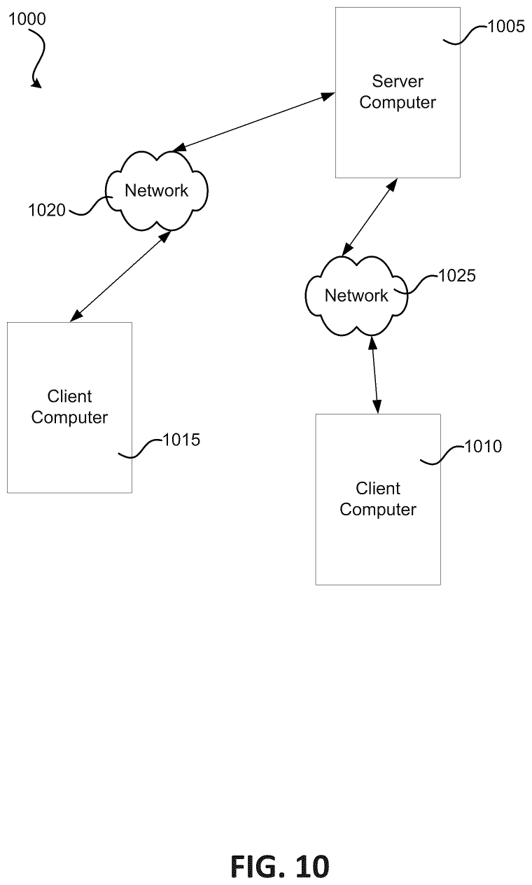

FIG. 10 illustrates one example of a diagrammatic illustration of an exemplary network disbursed implementation of an ECTPM system 1000. System 1000 has its components disbursed across physical components (e.g., memory elements, processors, etc.) of computer devices 1005, 1010, and 1015. Computer devices 1005, 1010, 1015 are connected via networks 1020, 1025. One example of a disbursed implementation of an ECTPM includes a portion of the ECTPM system stored in a memory of computing device 1005 for execution by a processor of device 1005 and communication with one or more of computers 1010, 1015 on which a portion (e.g., a client portion) of the ECTPM system is stored (e.g., via a memory) and executed (e.g., via a processor). In one such example, programmatic elements (such as occurrence, procedure, and cycle programmatic elements) are downloaded to a client portion from computer 1005 with executable programming running on a processor of computer 1005 and a displayable and interactive portion being run at the client portion for generation of iterations of cycles using an occurrence programmatic elements. In another example, a majority of an ECTPM system is stored and processed on computer 1005 with displayable interactions being communicated to one or more of client computers 1010, 1015. Examples of computing devices are discussed below with respect to FIG. 17.

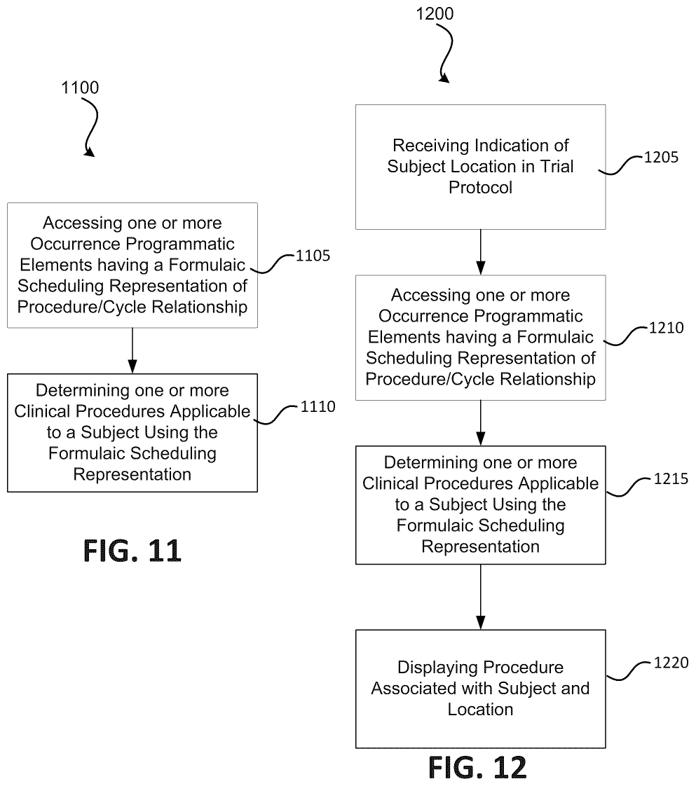

FIG. 11 illustrates one exemplary implementation of a method 1100 for interacting with a clinical trial protocol via an electronic clinical trial protocol management system. At step 1105, one or more occurrence programmatic elements in an electronic clinical trial protocol management system are accessed (e.g., via a processor and a memory element of an ECTPM system). In one exemplary implementation an occurrence programmatic element includes a formulaic representation of a scheduling of a clinical procedure within one or more treatment cycles where the formulaic representation includes information that relates one or more procedure programmatic elements (e.g., representing the clinical procedure) to one or more iterations of a treatment cycle programmatic element (e.g., representing a treatment cycle).

At step 1110, one or more clinical procedures applicable to a subject of a clinical trial represented in the ECTPM system via a clinical trial protocol are determined using one or more of the occurrence programmatic elements (e.g., as discussed above for relating a procedure programmatic element to a cycle programmatic element). In one such example, the one or more clinical procedures are applicable to the subject's location in a progression through the clinical trial protocol. For example, a subject may have completed procedure 3 of visit 2 of iteration 4 of cycle 1. Determination of an applicable clinical procedure in such an example may include using the occurrence programmatic element (and possibly some input from a user of the ECTPM system regarding whether the subject properly completed the procedure/visit/etc. and/or is eligible to move to a subsequent iteration of the cycle) to determine the next procedure for the subject in the protocol. In another example, an input of a subject name may be used to refer to data records that stored the last known location of the subject in the clinical trial and the occurrence programmatic element is accessed to determine that location and/or a next location in the trial for that subject. In yet another example, an input of a subject name and a desired historical location of that subject in a clinical trial can be utilized with the occurrence programmatic element to find that location and determine a procedure applicable to that location.

To determine a location of a subject in a clinical trial represented in an ECTPM system, an input from a user may be received. User inputs and related devices for inputting information to an ECTPM system are discussed herein. In one example, determining a location of a subject includes determining one or more of a treatment cycle, an iteration of a treatment cycle, and an interval of an iteration of a treatment cycle for the progression of the subject in the clinical trial. In another example, an indication of a location of a subject (including at least one or an information representing a treatment cycle, an iteration of a treatment cycle, and an interval of an iteration of a treatment cycle for the progression of the subject in the clinical trial) is received via an ECTPM system. In yet another example, a location of a subject in a clinical trial includes an indication of a treatment cycle, an iteration of a treatment cycle, and an interval of an iteration of a treatment cycle for the progression of the subject in the clinical trial. An indication of a location of a subject in a clinical trial in an ECTPM system can be received by an ECTPM system in a variety of ways that will be understood from the disclosure herein. Example ways to receive an indication of a location of a subject include, but are not limited to, data entry from a user (e.g., via a user input/interface element/device associated with the ECTPM system), data transfer to the ECTPM system (e.g., from a memory element, such as a removable memory element; from a network, etc.), from a memory within the ECTPM system (e.g., from data stored from a prior session of interacting with the subject records of an ECTPM system), and any combinations thereof.

As discussed above, an occurrence programmatic element may be part of a procedure programmatic element. In one example, at least one occurrence programmatic element is part of a procedure programmatic element. Also as discussed above, an occurrence programmatic element may be part of a cycle programmatic element. In one example, at least one occurrence programmatic element is part of a cycle programmatic element. It is noted that a treatment cycle programmatic element may be fully defined within a procedure programmatic element. In one example, at least one treatment cycle programmatic element of an ECTPM system is fully defined within a procedure programmatic element. It is also noted that a procedure programmatic element may be fully defined within a cycle programmatic element. In one example, at least one procedure programmatic element of an ECTPM system is fully defined within a cycle programmatic element.

One or more procedures for a subject at a particular location of a trial (e.g., those determined at step 1110 may be utilized in a variety of ways. Example ways to utilize the one or more procedures include, but are not limited to, displaying to a user (e.g., via a display element of a computer device of the ECTPM system) information related to or describing a procedure, outputting (e.g., printing, transferring over a network, etc.) information related to or describing a procedure, storing to a memory information related to or describing a procedure, analyzing (e.g., internally to the ECTPM system) information related to or describing a procedure, and any combinations thereof.

Also note that an ECTPM system may also similarly utilize information related to any other location (other than a procedure) within a clinical trial or programmatic element of the same. Example other locations include, but are not limited to, a visit, a cycle, a cycle iteration, a source document, an electronic copy of a source document, data entered into the system related to any of the same (e.g., clinical trial measurement data), and any combinations thereof.

FIG. 12 illustrates another exemplary implementation of a method 1200 for interacting with a clinical trial protocol via an electronic clinical trial protocol management system. Exemplary details, concepts, aspects, features, characteristics, examples, and/or alternatives discussed above with respect to method 1100 also apply here for corresponding items of method 1200 where appropriate and are not described again here for brevity sake. At step 1205, an indication of a subject location (e.g., tracking location) within a clinical trial of an ECTPM system is received via the ECTPM system. At step 1210, one or more occurrence programmatic elements in an electronic clinical trial protocol management system are accessed (e.g., via a processor and a memory element of an ECTPM system). In one exemplary implementation an occurrence programmatic element includes a formulaic representation of a scheduling of a clinical procedure within one or more treatment cycles where the formulaic representation includes information that relates one or more procedure programmatic elements (e.g., representing the clinical procedure) to one or more iterations of a treatment cycle programmatic element (e.g., representing a treatment cycle). At step 1215, one or more clinical procedures applicable to a subject of a clinical trial represented in the ECTPM system via a clinical trial protocol are determined using one or more of the occurrence programmatic elements (e.g., as discussed above for relating a procedure programmatic element to a cycle programmatic element). At step 1220, information related to at least one of the one or more select clinical procedures is displayed to a user of an ECTPM system. Other utilizations of the information may also occur as discussed above.

Another issue that arises in interactions with prior art electronic management systems is problems with tracking a clinical trial source document (“source document”) within regulatory limitations in a given system. Systems and methods for interacting with an ECTPM system are disclosed in which a source document is tracked in an ECTPM system. In such a ECTPM system, it is not necessary that particular structural elements of method steps described above with respect to any given details, concepts, aspects, features, characteristics, examples, and/or alternatives discussed above with respect to the treatment cycle and occurrence programmatic element are included in a source document tracking embodiment. However, where appropriate details, concepts, aspects, features, characteristics, examples, and/or alternatives of components and/or methodologies discussed above may apply to an ECTPM system utilized in tracking a source document.

Examples of a source document include, but are not limited to, notes of a clinician participating in a clinical trial (e.g., paper and/or electronically-generated original notes), notes of a subject participating in a clinical trial (e.g., paper, electronic), a document having results of a procedure of a clinical trial, a document having a measurement related to a clinical trial, a graph of a medical instrument (e.g., an EKG graph, an MRI result), a medical image document, a data file including information from a clinical trial, and any combinations thereof.

Example file formats for a clinical trial source document include, but are not limited to, a portable document format (PDF) (e.g., a PDF format of the Adobe Systems, Inc. of San Jose, California), a Joint Photographic Experts Group (JPG or JPEG) document format, a tagged image file format (TIFF) document, PNG, DICOM, ACR/NEMA, HL7, MPEG, AVI, WEBM, WEBP, WMV, MOV, QT, GIF, VOB, YUV, and any combinations thereof.

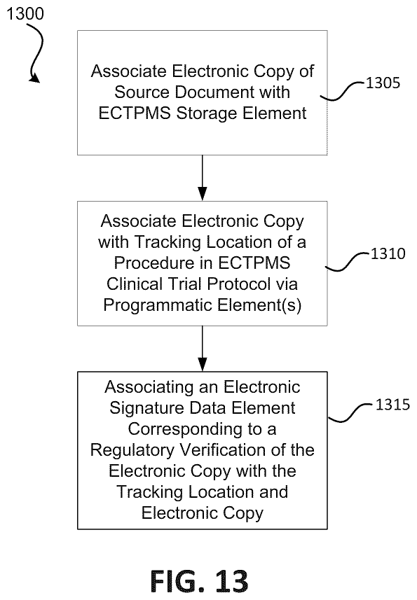

FIG. 13 illustrates yet another exemplary implementation of a method 1300 for interacting with a clinical trial protocol via an electronic clinical trial protocol management system. At step 1305, an electronic copy of a source document related to a clinical trial with respect to a subject of the clinical trial is associated with a storage element of an ECTPM system. In one example, an associating of an electronic copy of a source document includes storing the electronic copy in one or more memories of the ECTPM system. Optionally, the source document itself may also be associated/stored with a memory of the ECTPM system. A non-electronic form source document may be made into an electronic form for the electronic copy (e.g., via optical scanning). An electronic form of a source document may be made into an electronic copy that has a different format than the original format of the source document.

At step 1310, the electronic copy is associated with a tracking location representing a location in the clinical trial protocol related to the corresponding source document. In one example, a tracking location corresponds to a location in the clinical trial protocol of a clinical procedure related to the source document. An associating with a tracking location may include association to at least one of a programmatic element representing at least one of a visit, a clinical procedure, a treatment cycle, an interval of a treatment cycle, and an iteration of a treatment cycle.

Example locations (“tracking locations”) within an electronic clinical trial protocol management system include, but are not limited to, a protocol, a site, a subject, a visit, a cycle programmatic element, a cycle iteration, a procedure programmatic element, a visit programmatic element, measurement, clinical event, data entry, and any combinations thereof. In one example an association with a tracking location includes an association to a corresponding programmatic element to the tracking location. An associating can occur by any structure or programmatic code that allows for identification by the ECTPM system of the location and the corresponding electronic copy of the source document (and, optionally the source document itself). Example ways of associating include, but are not limited to, using a location pointer (e.g., in a data element, in a programmatic element, in a file attached to the electronic copy, in the electronic copy file itself, etc.), tagging (e.g., in a data element, in a programmatic element, in a file attached to the electronic copy, in the electronic copy file itself, etc.), storing the electronic copy with a correlation to the location, a link (e.g., in a data element, in a programmatic element, in a file attached to the electronic copy, in the electronic copy file itself, etc.), use of a non-SQL data file, a database entry in a table, a database entry in a programmatic coding, and any combinations thereof. Example location pointers for an electronic copy of a source document (these are the pointers into the protocol) include, but are not limited to, a protocol ID, a site ID, a subject ID, a visit ID, a cycle programmatic element ID, a cycle iteration ID, a procedure programmatic element ID, a visit programmatic element ID, measurement ID, clinical event ID, data entry ID, and any combinations thereof.

At step 1315, an electronic signature data element representing a signature of a user of the ECTPM system is associated with the electronic copy (and optionally directly with the tracking location). An associating can occur via a similar way as associating a tracking location with an electronic copy. In one example, an electronic signature data element results from an action by a user in association with a verification of the electronic copy (e.g., a verification related to a regulatory requirement). An electronic signature data element is any data file and/or document that can be stored in a memory of an ECTPM system (e.g., image file, scan of an actual signature, e-verification file with an electronic signature, etc.).

A certification statement may be included with an electronic signature (e.g., stored in the ECTPM system and associated with the electronic signature data element and/or the electronic copy of a source document corresponding to the signature). Such a certification statement may be displayed to a user that is associating an electronic copy of a source document with a tracking location and providing an electronic signature (e.g., as per step 1315). In one example, a certification statement is related to a regulatory requirement for producing a certified electronic copy of a clinical trial source document.

In one example of method 1300, an electronic copy of a source document is deconstructed into one or more individual displayable pages. Example ways of deconstructing to one or more individual displayable pages include, but are not limited to, rasterizing each page into a file (e.g., an image file), use of a sprite file (e.g., multiple pages on an image with electronic instructions mapping each page to a displayable image), including multiple individual displayable images in on electronic document file (e.g., a TIFF file), separate data files for each page (e.g., separate PNG files, separate PDF files), including data instructions with an electronic copy defining individual displayable images, and any combinations thereof. Individual displayable page images can be stored as above and associated with a tracking location and a signature data element. In one example, each individual displayable page image can be associated with the same or different signature data element.

Clinical trial data and information may also be associated with a tracking location. Clinical trial data (e.g., data entered into an ECTPM system) may include attributes including, but not limited to, a value, a unit, a normal range (low/high, greater than, less than), a clinical significance, an associated entry of a log for an event, a done/not done for measurement indication, and any combinations thereof.

Example file formats for an electronic copy of a clinical trial source document include, but are not limited to, a portable document format (PDF) (e.g., a PDF format of the Adobe Systems, Inc. of San Jose, California), a Joint Photographic Experts Group (JPG or JPEG) document format, a tagged image file format (TIFF) document, PNG, DICOM, ACR/NEMA, HL7, MPEG, AVI, WEBM, WEBP, WMV, MOV, QT, GIF, VOB, YUV, and any combinations thereof.

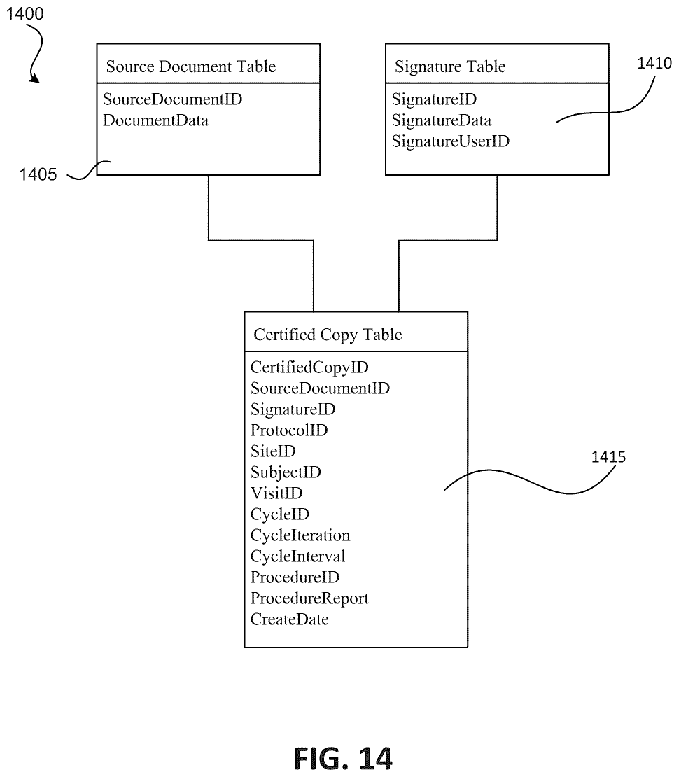

FIG. 14 includes an illustration 1400 of an exemplary association amongst a source document (represented in the SourceDocument Table 1405), an electronic copy of a source document (represented in CertifiedCopy Table 1410), and an electronic signature data element (represented in Signature Table 1415). Tables 1405, 1410, 1415 are organizations of data in an ECTPM system, such as any of the systems disclosed herein. The source document representation has a Source Document ID (e.g., a pointer for the source document, a pointer to the source document stored in a memory, etc.) and Document Data in the SourceDocument Table. Signature Table includes a Signature ID (e.g., a pointer for the electronic signature data element, a pointer to the electronic signature data element in a memory, etc.), Signature Data, and a Signing User ID (a pointer to information and/or a file related to the user of the ECTPM system that signs the electronic signature). The CertifiedCopy Table includes a Certified Copy ID ((e.g., a pointer for the certified copy document, a pointer to the certified copy document stored in a memory, etc.), the SourceDocument ID (associating the source document to the electronic copy of the document), and the Signature ID (associating the electronic signature data element to the electronic copy of the document and, indirectly, to the source document). The CertifiedCopy Table also includes a Protocol ID (a pointer to a protocol), a Site ID (a reference to the clinical trial site programmatic element), a subject ID (a reference identifier for the subject related to the source document), a visit ID (a reference to a visit programmatic element), a cycle ID (a reference to a cycle programmatic element), a cycle iteration (a reference to the iteration of the cycle related to the source document), a procedure ID (a pointer to the procedure programmatic element related to the source document), a procedure repeat reference, and a create date for the creation of the electronic copy document.

Typically, in traditional health-related Electronic Data Capture (EDC) systems for clinical trials and Electronic Medical Record (EMR) systems that are used to record and manage patient records, there is no functionality for relating electronic copies of a source document (e.g., a medical record document associated with patient care), let alone certified electronic copies (e.g., a copy including a signature and certification required by regulatory rules), with electronic data that is collected for purposes of a clinical trial. EDC systems are generally separate systems from EMR systems. In some instances, a clinical trial may take place at a clinical site that also has regular medical clients. Such a site may have an EMR system in which it keeps its regular patient medical records. Regular medical treatment for a patient may overlap (e.g., be the same treatment) with protocol procedures for a clinical trial (e.g. where the subject of the clinical trial is also a regular patient of the clinical site). A clinician that is participating in a clinical trial may still keep all of the clinical trial subject's regular medical records in the clinical site's normal EMR system. At some point (for example, at the time of delivery of medical care that overlaps with clinical trial protocol procedures) the doctor may enter data into the clinical site's EMR system. Data for a clinical trial protocol is typically kept in an EDC system that is customized for the particular clinical trial protocol (e.g., in the prior art via long form encoding of data representations and code to fit the protocol). Clinical site personnel may need to enter data and information that overlaps between the regular medical treatment (input into the EMR) and the clinical trial (required to be in the EDC system) into each system at different times. In some examples, clinical site personnel may attempt to enter the information into the EMR and the EDC at times that are closer to each other (e.g., both at the time of delivery of medical care to the patient). However, this involves having the two separate systems (EMR and EDC) open on one or more computers and switching between the two to allow for entry.

Medical source documents may complicate record-keeping for clinical trials even further. In some examples, source documents may be stored in a clinical site's EMR system. Example locations where a source document may be stored include, but are not limited to, in an EMR system, in a paper record, in an electronic system associated with a medical device (e.g., a medical imaging device), in an electronic system associated with a general data store (e.g., a data storage on a server, a data storage on a clinician computer, etc.), in a physical file, in a clinician notebook, and any combinations thereof. In some examples, different types of source documents may be stored in different locations. During an audit of a clinical trial protocol, source documents must be retrieved from their storage locations and may require manual correlation to procedures and clinical trial data in the EDC for the clinical trial.

An ECTPM system of the current disclosure (e.g., one configured to have an electronic copy of a source document associated with a particular location in a clinical trial protocol such as is described in method 1300 above) may include one or more programmatic elements and one or more functionalities that work together to allow simultaneous display (e.g., side-by-side) of a clinical trial data entry portion and an electronic copy of a source document portion. Such a combined display may be a graphical user interface for display to a user of the ECTPM system (e.g., via a display device of a computing system associated with the ECTPM system). A source document electronic copy display portion of a combined display is configured to display at least a portion of an electronic copy of a source document (e.g., an electronic copy associated with a tracking location in a clinical trial protocol within an ECTPM system). A clinical trial data entry portion of a combined display is configured to provide a user with a display interface for entry of one or more data element and/or information that is/are related to (a) the source document electronic copy displayed in the corresponding source document electronic copy display portion of the combined display and/or (b) the tracking location to which the source document electronic copy is associated. Display configuration of a source document electronic copy may include a scroll or other display shifting mechanism to allow for changing the view of the displayed portion of the source document electronic copy. Various data entry configuration will be understood by those of ordinary skill in the art. Example data entry elements for a configuration of a data entry display portion include, but are not limited to, a text entry element, a toggle button element, a pull-down menu element, a check box element, and any combinations thereof. A data entry display portion may include labels and/or other information for guiding entry of particular information. In one exemplary aspect, a user providing entry via a combined display may utilize data and/or information from the display of the source document display portion to assist with entry of data and/or information for the clinical trial via a data entry element of a data entry display portion.

Any one or more of the programmatic elements and functionalities discussed above with respect to the various examples and implementations of an ECTPM system may be employed to link the electronic copy of the source document (and its corresponding display in a combined display) to (a) a tracking location in a clinical trial protocol (e.g., as discussed above with respect to FIG. 13, via use of programmatic elements of the ECTPM system, etc.)) and/or (b) to clinical trial data and/or information entered via a data entry display portion of a combined display. In one example, a tracking location to which clinical trial data and/or an electronic copy of a source document are associated is a location in an iteration of a cycle (e.g., association via a cycle programmatic element, an iteration of a treatment cycle, an occurrence programmatic element, a procedure programmatic element, and/or other programmatic element of an ECTPM system described above). Other tracking location examples include, but are not limited to, a treatment cycle, an iteration of a treatment cycle, and an interval of an iteration of a treatment cycle, a procedure of a clinical trial, a visit of a clinical trial, and any combinations thereof.

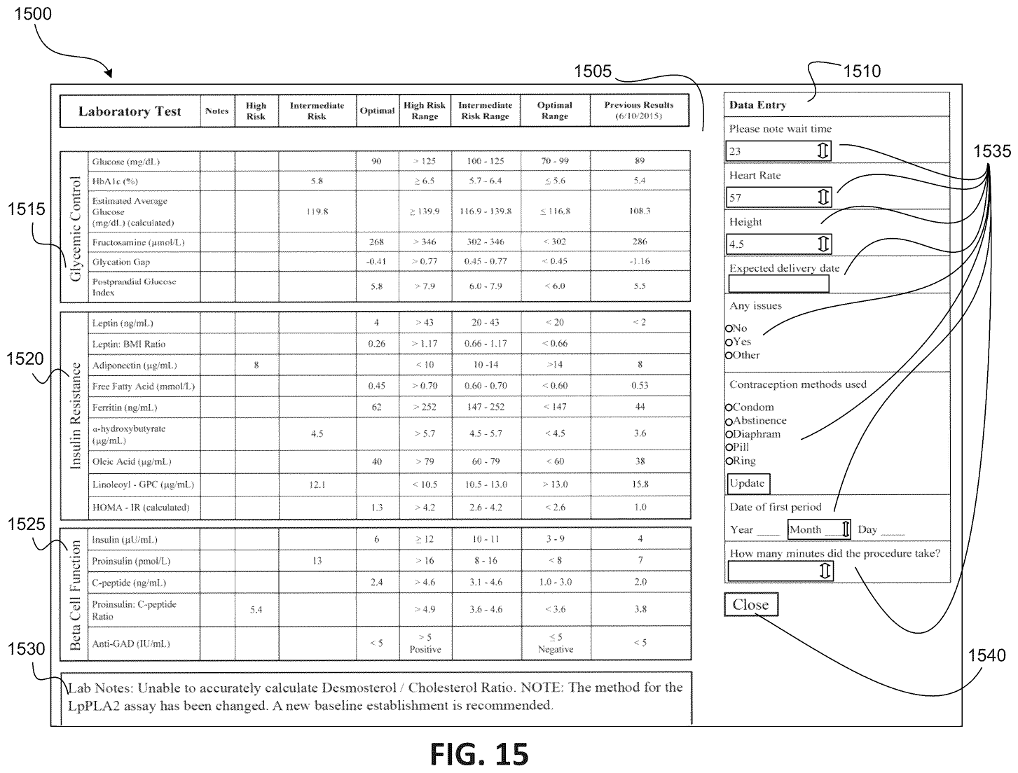

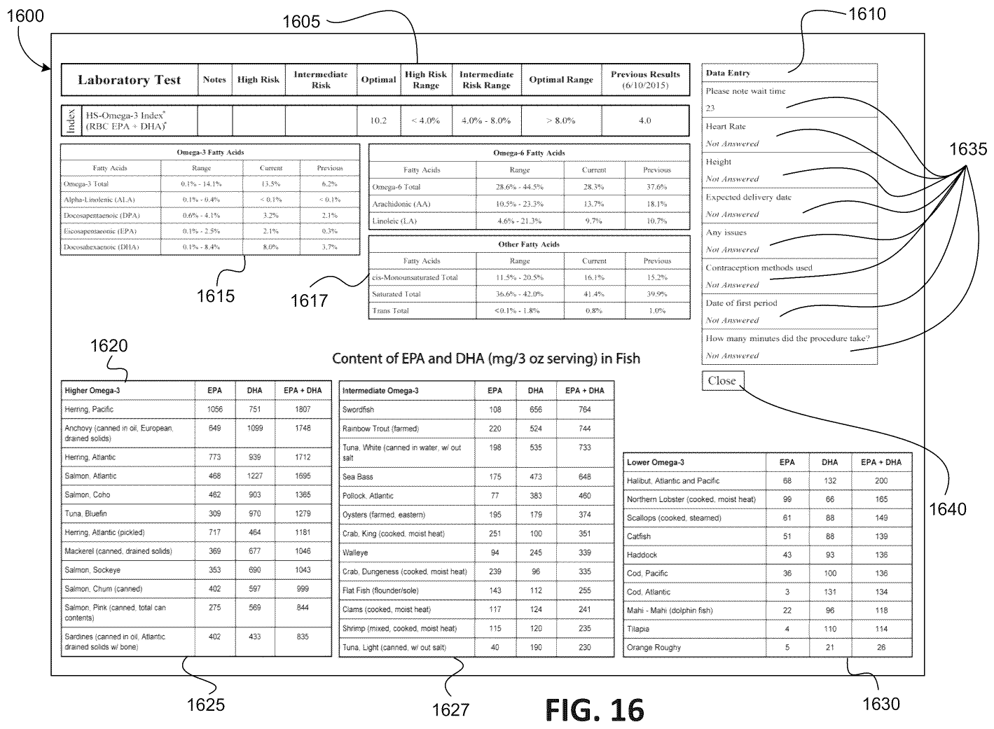

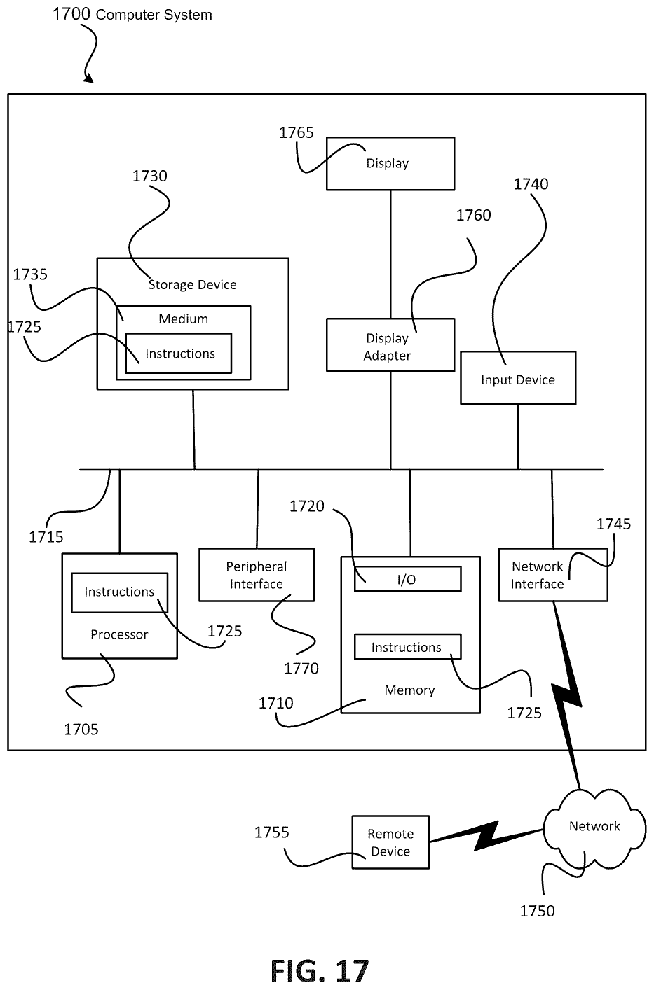

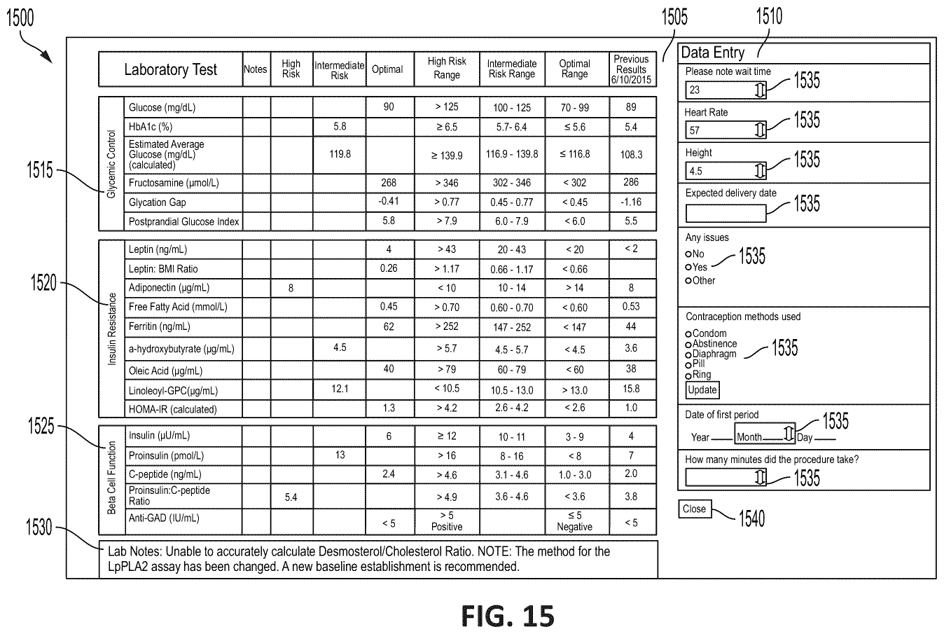

FIG. 15 illustrates one exemplary implementation of a combined display 1500 of a source document electronic copy and clinical trial data entry. Combined display 1500 includes a source document electronic copy display portion 1505 and a clinical trial data entry portion 1510. In this display a dark graphical border divides portion 1505 and 1510. In one example, the electronic copy of a source document displayed via a source document electronic copy display portion 1505 is a certified electronic copy (e.g., one entered to a corresponding ECTPM system via a method such as method 1300). Electronic copies are discussed in detail above. The electronic copy of a source document displayed in portion 1505 is an electronic copy of a laboratory test for a subject of a clinical trial associated with an ECTPM system. The laboratory test includes test results in tables 1515, 1520, 1525 and a notes section 1530. Data entry portion 1510 includes multiple data entry elements 1535 for entering data and information related to the subject of the clinical trial that is associated with the source document represented by the electronic copy displayed in portion 1505. Data and/or information entered via portion 1510 may include data and/or information directly from the electronic copy of a source document and/or from another source (e.g., from an interaction with a subject associated with the electronic copy). In one example, the source document represented in portion 1505 is associated with the same tracking location in the clinical trial protocol as the data entered via portion 1510 (e.g., a laboratory test that is related to a clinical visit at which other information is obtained by a clinician an entered into data entry elements of the display). A button 1540 is configured to allow a user to close the combined display 1500.

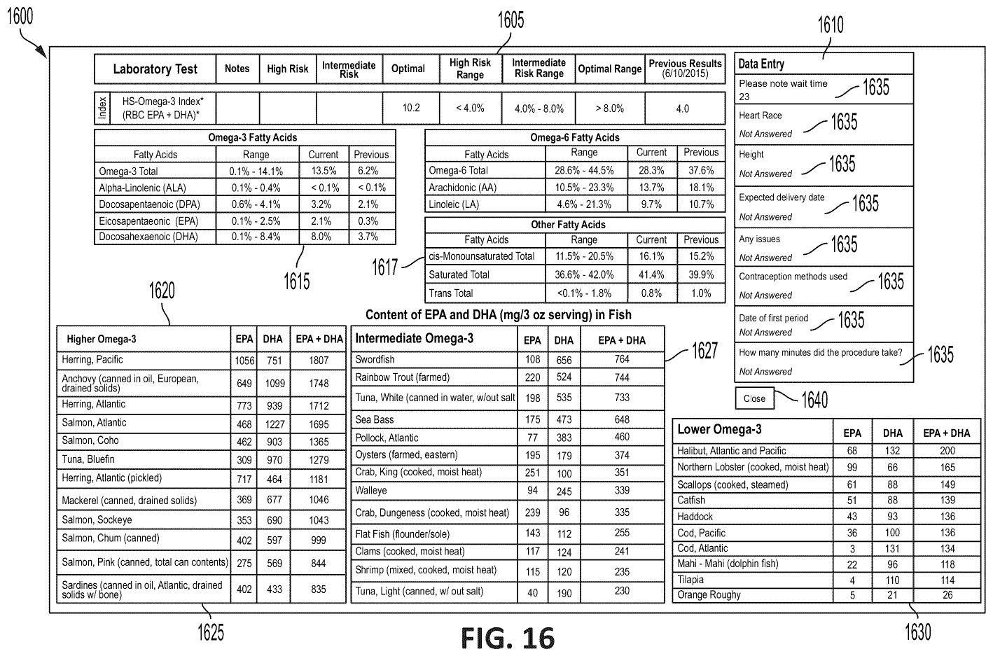

FIG. 16 illustrates another exemplary implementation of a combined display 1600 of a source document electronic copy and clinical trial data entry. Combined display 1600 includes a source document electronic copy display portion 1605 and a clinical trial data entry portion 1610. In this display a dark graphical border divides portion 1605 and 1610. In one example, the electronic copy of a source document displayed via a source document electronic copy display portion 1605 is a certified electronic copy (e.g., one entered to a corresponding ECTPM system via a method such as method 1300). Electronic copies are discussed in detail above. The electronic copy of a source document displayed in portion 1605 is an electronic copy of a laboratory test for a subject of a clinical trial associated with an ECTPM system. The laboratory test includes test results in tables 1615, 1617, 1620, 1623, 1625, 1627, and 1630. Data entry portion 1610 includes multiple data entry elements 1635 for entering data and information related to the subject of the clinical trial that is associated with the source document represented by the electronic copy displayed in portion 1605. Data and/or information entered via portion 1610 may include data and/or information directly from the electronic copy of a source document and/or from another source (e.g., from an interaction with a subject associated with the electronic copy). In one example, the source document represented in portion 1605 is associated with the same tracking location in the clinical trial protocol as the data entered via portion 1610 (e.g., a laboratory test that is related to a clinical visit at which other information is obtained by a clinician an entered into data entry elements of the display). A button 1640 is configured to allow a user to close the combined display 1600.

The following are exemplary implementations showing aspects of the concepts discussed above:

### Example 1

A computer-implemented method of monitoring a clinical trial protocol via an electronic clinical trial protocol management system, the electronic clinical trial protocol management system being programmed with information describing a clinical trial protocol for a clinical trial having one or more treatment cycles, the electronic clinical trial protocol management system including one or more procedure programmatic elements and one or more treatment cycle programmatic elements, the method including receiving via the electronic clinical trial protocol management system an indication of a first position of a subject in the clinical trial, the first position representing at least one of a treatment cycle, an iteration of a treatment cycle, and an interval of an iteration of a treatment cycle for the progression of the subject in the clinical trial; accessing the one or more procedure programmatic elements in the electronic clinical trial protocol management system, each of the one or more procedure programmatic elements including information defining one or more characteristics of a clinical procedure related to the clinical trial, at least one procedure programmatic element of the one or more procedure programmatic elements including one or more cycle occurrence definitions, the one or more cycle occurrence definitions including information related to at least one of the one or more treatment cycle programmatic elements and information for scheduling within the one or more treatment cycles the clinical procedure corresponding to the at least one procedure programmatic element; determining one or more clinical procedures applicable to the subject for the first position using the one or more procedure programmatic elements; displaying information related to at least one of the one or more clinical procedures to a user of the electronic clinical trial protocol management system.

The receiving and indication of a first position may include determining the treatment cycle, iteration of the treatment cycle, and the interval of the iteration of the treatment cycle for the progression of the subject in the clinical trial. The at least one cycle occurrence definition may include a formulaic representation of the scheduling of a clinical procedure within one or more treatment cycles. The formulaic representation may be configured to allow the electronic clinical trial protocol management system to model a first number of treatment cycle iterations for the subject without modification to the one or more procedure programmatic elements or the one or more treatment cycle programmatic elements, the first number of treatment cycle iterations not being known at the time that the one or more procedure programmatic elements or the one or more treatment cycle programmatic elements are programmed into the electronic clinical trial protocol management system. The formulaic representation may also include one or more characteristic information elements selected from the group consisting of an indicator of interval for implementation of a procedure, an indicator of a starting cycle iteration for implementation of a procedure, an indicator of an ending cycle iteration for implementation of a procedure, and an indicator of cycle iterations to skip implementation of a procedure. The at least one cycle occurrence definition may include one or more characteristic information elements selected from the group consisting of an indicator of interval for implementation of a procedure, an indicator of a starting cycle iteration for implementation of a procedure, an indicator of an ending cycle iteration for implementation of a procedure, and an indicator of cycle iterations to skip implementation of a procedure. The one or more procedure programmatic elements and the one or more treatment cycle programmatic elements may be in a data format selected from the group consisting of an object-oriented data format, a tabular data format, a JavaScript Object Notation (JSON) format, an extensible markup language (XML) format, and any combinations thereof. The at least one of the one or more treatment cycle programmatic elements may be defined fully within at least one of the one or more procedure programmatic elements. The at least one of the one or more treatment cycle programmatic elements may be defined as a separate data element that is referenced by a cycle occurrence definition of the one or more cycle occurrence definitions. The at least one of the one or more cycle occurrence definitions may include information related to a clinical trial visit programmatic element. The at least one of the one or more treatment cycle programmatic elements may include a formulaic definition of an interval window and the treatment cycle iteration for the application of the interval window, the formulaic definition including information for a period of time before an interval and/or a period of time after an interval in which a subject may have a clinical trial visit compliant with the interval. The at least one of the one or more treatment cycle programmatic elements may include information defining a minimum number of iterations for the corresponding treatment cycle and/or a maximum number of iterations for the corresponding treatment cycle.

### Example 2

A computer-implemented method of interacting with a clinical trial protocol via an electronic clinical trial protocol management system, the electronic clinical trial protocol management system being programmed with information describing a clinical trial protocol for a clinical trial and including one or more programmatic elements representing at least one of a visit, a clinical procedure, a treatment cycle, an interval of a treatment cycle, and an iteration of a treatment cycle, the method including: storing a first electronic copy of a clinical trial source document in a storage element of an electronic clinical trial protocol management system, the clinical trial source document being related to a clinical procedure performed with respect to a subject of the clinical trial; associating the first electronic copy with a first tracking location within the electronic clinical trial protocol management system, the first tracking location representing a location in the clinical trial protocol of the clinical procedure, the associating including an association to at least one of the one or more programmatic elements; and associating an electronic signature data element representing a signature of a user of the electronic clinical trial protocol management system with the first electronic copy, the electronic signature resulting from an action by the user in association with a verification of the first electronic copy, the verification related to a regulatory requirement.

It is to be noted that any one or more of the aspects and embodiments described herein may be conveniently implemented using one or more machines (e.g., one or more computing devices that are utilized as a user computing device for an electronic document, one or more server devices, such as a document server) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk (e.g., a conventional floppy disk, a hard drive disk), an optical disk (e.g., a compact disk “CD”, such as a readable, writeable, and/or re-writable CD; a digital video disk “DVD”, such as a readable, writeable, and/or rewritable DVD), a magneto-optical disk, a read-only memory “ROM” device, a random access memory “RAM” device, a magnetic card, an optical card, a solid-state memory device (e.g., a flash memory), an EPROM, an EEPROM, and any combinations thereof. Such examples are hardware storage media. A machine-readable medium, as used herein, is intended to include a single medium as well as a collection of physically separate media, such as, for example, a collection of compact disks or one or more hard disk drives in combination with a computer memory. As used herein, a machine-readable storage medium does not include a signal.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a personal digital assistant “PDA”, a mobile telephone, a Smartphone, etc.), a web appliance, a network router, a network switch, a network bridge, any machine capable of executing a sequence of instructions that specify an action to be taken by that machine, and any combinations thereof. In one example, a computing device may include and/or be included in, a kiosk.

FIG. 17 shows a diagrammatic representation of one embodiment of a computing device in the exemplary form of a computer system 1700 within which a set of instructions for causing the device to perform any one or more of the aspects and/or methodologies of the present disclosure may be executed. It is also contemplated that multiple computing devices may be utilized to implement a specially configured set of instructions for causing the device to perform any one or more of the aspects and/or methodologies of the present disclosure. Any such device or combination of devices constitutes a specially programmed machine for the functionalities of the current disclosure (e.g., an electronic clinical trial protocol management systems). Computer system 1700 includes a processor 1705 and a memory 1710 that communicate with each other, and with other components, via a bus 1715. Bus 1715 may include any of several types of bus structures including, but not limited to, a memory bus, a memory controller, a peripheral bus, a local bus, and any combinations thereof, using any of a variety of bus architectures.

Memory 1710 may include various components (e.g., machine readable media) including, but not limited to, a random access memory component (e.g., a static RAM “SRAM”, a dynamic RAM “DRAM”, etc.), a read only component, and any combinations thereof. In one example, a basic input/output system 1720 (BIOS), including basic routines that help to transfer information between elements within computer system 1700, such as during start-up, may be stored in memory 1710. Memory 1710 may also include (e.g., stored on one or more machine-readable media) instructions (e.g., software) 1725 embodying any one or more of the aspects and/or methodologies of the present disclosure. In another example, memory 1710 may further include any number of program modules including, but not limited to, an operating system, one or more application programs, other program modules, program data, and any combinations thereof.

Computer system 1700 may also include a storage device 1730. Examples of a storage device (e.g., storage device 1730) include, but are not limited to, a hard disk drive for reading from and/or writing to a hard disk, a magnetic disk drive for reading from and/or writing to a removable magnetic disk, an optical disk drive for reading from and/or writing to an optical media (e.g., a CD, a DVD, etc.), a solid-state memory device, and any combinations thereof. Storage device 1730 may be connected to bus 1715 by an appropriate interface (not shown). Example interfaces include, but are not limited to, SCSI, advanced technology attachment (ATA), serial ATA, universal serial bus (USB), IEEE 1794 (FIREWIRE), and any combinations thereof. In one example, storage device 1730 (or one or more components thereof) may be remotely interfaced with computer system 1700 (e.g., via an external port connector (not shown)). Particularly, storage device 1730 and an associated machine-readable medium 1735 may provide nonvolatile and/or volatile storage of machine-readable instructions, data structures, program modules, and/or other data for computer system 1700. In one example, software 1725 may reside, completely or partially, within machine-readable medium 1735. In another example, software 1725 may reside, completely or partially, within processor 1705.

Computer system 1700 may also include an input device 1740. In one example, a user of computer system 1700 may enter commands and/or other information into computer system 1700 via input device 1740. Examples of an input device 1740 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), touchscreen, and any combinations thereof. Input device 1740 may be interfaced to bus 1715 via any of a variety of interfaces (not shown) including, but not limited to, a serial interface, a parallel interface, a game port, a USB interface, a FIREWIRE interface, a direct interface to bus 1715, and any combinations thereof. Input device may include a touch screen interface that may be a part of or separate from display 1765, discussed further below.

A user may also input commands and/or other information to computer system 1700 via storage device 1730 (e.g., a removable disk drive, a flash drive, etc.) and/or a network interface device 1745. A network interface device, such as network interface device 1745 may be utilized for connecting computer system 1700 to one or more of a variety of networks, such as network 1750, and one or more remote devices 1755 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1750, may employ a wired and/or a wireless mode of communication. In general, any network topology may be used. Information (e.g., data, software 1725, etc.) may be communicated to and/or from computer system 1700 via network interface device 1745.

Computer system 1700 may further include a video display adapter 1760 for communicating a displayable image to a display device, such as display device 1765 (e.g., for providing user access to one or more user interfaces. Examples of a display device include, but are not limited to, a liquid crystal display (LCD), a cathode ray tube (CRT), a plasma display, a light emitting diode (LED) display, and any combinations thereof. In addition to a display device, a computer system 1700 may include one or more other peripheral output devices including, but not limited to, an audio speaker, a printer, and any combinations thereof. Such peripheral output devices may be connected to bus 1715 via a peripheral interface 1770. Examples of a peripheral interface include, but are not limited to, a serial port, a USB connection, a FIREWIRE connection, a parallel connection, and any combinations thereof.

Some of the details, concepts, aspects, features, characteristics, examples, and/or alternatives of a component/element discussed above with respect to one implementation, embodiment, and/or methodology may be applicable to a like component in another implementation, embodiment, and/or methodology, even though for the sake of brevity it may not have been repeated above. It is noted that any suitable combinations of components and elements of different implementations, embodiments, and/or methodologies (as well as other variations and modifications) are possible in light of the teachings herein, will be apparent to those of ordinary skill, and should be considered as part of the spirit and scope of the present disclosure. Additionally, functionality described with respect to a single component/element is contemplated to be performed by a plurality of like components/elements (e.g., in a more dispersed fashion locally and/or remotely). Functionality described with respect to multiple components/elements may be performed by fewer like or different components/elements (e.g., in a more integrated fashion).

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1.-17. (canceled)

18. A computer-implemented method of interacting with a clinical trial protocol via an electronic clinical trial protocol management system, the electronic clinical trial protocol management system being programmed with information describing a clinical trial protocol for a clinical trial having one or more treatment cycles, the electronic clinical trial protocol management system including one or more programmatic elements that model a number of treatment cycle iterations for a subject progressing through the clinical trial, the method comprising:
associating an electronic copy of a clinical trial source document with a tracking location in the clinical trial protocol within the electronic clinical trial protocol management system, wherein the clinical trial source document relates to a clinical procedure performed with respect to a subject of the clinical trial, and associating the electronic copy of the clinical trial source document with the tracking location comprises:
linking the electronic copy of the clinical trial source document to the tracking location via at least one programmatic element representing at least one of a visit, the clinical procedure, a treatment cycle, an iteration of a treatment cycle, and an interval of an iteration of a treatment cycle for the progression of the subject in the clinical trial;
generating a graphical user interface (GUI) including a first portion and a second portion;
displaying, via the first portion of the GUI, at least a portion of the electronic copy of the clinical trial source document;
displaying, via the second portion of the GUI and simultaneously with the displaying of the portion of the electronic copy of the clinical trial source document in the first portion, one or more GUI elements for receiving input of clinical trial information related to the portion of the electronic copy displayed in the first portion and/or the tracking location to which the electronic copy is associated within the electronic clinical trial protocol management system; and
associating the clinical trial information input via the one or more GUI elements in the second portion of the GUI to the tracking location to which the electronic copy is associated, wherein associating the clinical trial information input via the one or more GUI elements in the second portion of the GUI to the tracking location comprises linking the clinical trial information to the tracking location via the at least one programmatic element representing at least one of the visit, the clinical procedure, the treatment cycle, the iteration of a treatment cycle, and the interval of an iteration of a treatment cycle for the progression of the subject in the clinical trial.

19. The computer-implemented method of claim 18, wherein the one or more programmatic elements are in a data format that includes at least one of an object-oriented data format, a tabular data format, a JavaScript Object Notation (JSON) format, an extensible markup language (XML) format.

20. The computer-implemented method of claim 18, further comprising:
generating a certified electronic copy of the clinical trial source document, wherein generating the certified electronic copy comprises:
associating an electronic signature data element representing a signature of a user of the electronic clinical trial protocol management system with the electronic copy, the electronic signature resulting from an action by the user in association with a verification of the electronic copy, and
generating a data structure, in the memory of the electronic clinical trial protocol management system, linking the electronic signature data element with the electronic copy of the clinical trial source document.

21. The computer-implemented method of claim 20, further comprising:
displaying, via the first portion of the GUI, at least a portion of the certified electronic copy of the clinical trial source document.

22. The computer-implemented method of claim 20, wherein the verification is related to a regulatory requirement.

23. The computer-implemented method of claim 22, wherein generating the certified electronic copy further comprises:
associating a certification statement related to the regulatory requirement with the electronic signature data element and/or the electronic copy of the clinical trial source document.

24. The computer-implemented method of claim 20, further comprising:
deconstructing the electronic copy of the clinical trial source document into one or more displayable pages; and
associating the electronic signature data element representing the signature of the user of the electronic clinical trial protocol management system with each page of the one or more displayable pages.

25. An electronic clinical trial protocol management system programmed with information describing a clinical trial protocol for a clinical trial having one or more treatment cycles, the electronic clinical trial protocol management system including one or more programmatic elements that model a number of treatment cycle iterations for a subject progressing through the clinical trial, the electronic clinical trial protocol management system comprising:
at least one processor; and
at least one machine readable hardware storage medium containing machine executable instructions that when executed by the at least one processor cause the at least one processor to implement a method of interacting with a clinical trial protocol via the electronic clinical trial protocol management system, the method comprising:
associating an electronic copy of a clinical trial source document with a tracking location in the clinical trial protocol within the electronic clinical trial protocol management system, wherein the clinical trial source document relates to a clinical procedure performed with respect to a subject of the clinical trial, and associating the electronic copy of the clinical trial source document with the tracking location comprises:
associating the electronic copy of the clinical trial source document to at least one programmatic element representing at least one of a visit, the clinical procedure, a treatment cycle, an iteration of a treatment cycle, and an interval of an iteration of a treatment cycle for the progression of the subject in the clinical trial;
generating a graphical user interface (GUI) including a first portion and a second portion;
displaying, via the first portion of the GUI, at least a portion of the electronic copy of the clinical trial source document;
displaying, via the second portion of the GUI and simultaneously with the displaying of the portion of the electronic copy of the clinical trial source document in the first portion, one or more GUI elements for receiving input of clinical trial information related to the portion of the electronic copy displayed in the first portion and/or the tracking location to which the electronic copy is associated within the electronic clinical trial protocol management system; and
associating the clinical trial information input via the one or more GUI elements in the second portion of the GUI to the tracking location to which the electronic copy is associated, wherein associating the clinical trial information input via the one or more GUI elements in the second portion of the GUI to the tracking location comprises linking the clinical trial information to the tracking location via the at least one programmatic element representing at least one of the visit, the clinical procedure, the treatment cycle, the iteration of a treatment cycle, and the interval of an iteration of a treatment cycle for the progression of the subject in the clinical trial.

26. The electronic clinical trial protocol management system of claim 25, wherein the method further comprises:
generating a certified electronic copy of the clinical trial source document, wherein generating the certified electronic copy comprises:
associating an electronic signature data element representing a signature of a user of the electronic clinical trial protocol management system with the electronic copy, the electronic signature resulting from an action by the user in association with a verification of the electronic copy, and
generating a data structure, in the memory of the electronic clinical trial protocol management system, linking the electronic signature data element with the electronic copy of the clinical trial source document.

27. The electronic clinical trial protocol management system of claim 26, wherein the method further comprises:
displaying, via the first portion of the GUI, at least a portion of the certified electronic copy of the clinical trial source document.

28. The electronic clinical trial protocol management system of claim 26, wherein the verification is related to a regulatory requirement.

29. The electronic clinical trial protocol management system of claim 28, wherein generating the certified electronic copy further comprises:
associating a certification statement related to the regulatory requirement with the electronic signature data element and/or the electronic copy of the clinical trial source document.

30. The electronic clinical trial protocol management system of claim 26, wherein the method further comprises:
deconstructing the electronic copy of the clinical trial source document into one or more displayable pages; and
associating the electronic signature data element representing the signature of the user of the electronic clinical trial protocol management system with each page of the one or more displayable pages.

31. A machine readable hardware storage medium containing machine executable instructions that when executed by at least one processor of an electronic clinical trial protocol management system cause the at least one processor to implement a method of interacting with a clinical trial protocol via an electronic clinical trial protocol management system, the electronic clinical trial protocol management system being programmed with information describing a clinical trial protocol for a clinical trial having one or more treatment cycles, the electronic clinical trial protocol management system including one or more programmatic elements that model a number of treatment cycle iterations for a subject progressing through the clinical trial, the method comprising:
associating an electronic copy of a clinical trial source document with a tracking location in the clinical trial protocol within the electronic clinical trial protocol management system, wherein the clinical trial source document relates to a clinical procedure performed with respect to a subject of the clinical trial, and associating the electronic copy of the clinical trial source document with the tracking location comprises:
associating the electronic copy of the clinical trial source document to at least one programmatic element representing at least one of a visit, the clinical procedure, a treatment cycle, an iteration of a treatment cycle, and an interval of an iteration of a treatment cycle for the progression of the subject in the clinical trial;
generating a graphical user interface (GUI) including a first portion and a second portion;
displaying, via the first portion of the GUI, at least a portion of the electronic copy of the clinical trial source document;
displaying, via the second portion of the GUI and simultaneously with the displaying of the portion of the electronic copy of the clinical trial source document in the first portion, one or more GUI elements for receiving input of clinical trial information related to the portion of the electronic copy displayed in the first portion and/or the tracking location to which the electronic copy is associated within the electronic clinical trial protocol management system; and
associating the clinical trial information input via the one or more GUI elements in the second portion of the GUI to the tracking location to which the electronic copy is associated, wherein associating the clinical trial information input via the one or more GUI elements in the second portion of the GUI to the tracking location comprises linking the clinical trial information to the tracking location via the at least one programmatic element representing at least one of the visit, the clinical procedure, the treatment cycle, the iteration of a treatment cycle, and the interval of an iteration of a treatment cycle for the progression of the subject in the clinical trial.

32. The machine readable hardware storage medium of claim 31, wherein the method further comprises:
generating a certified electronic copy of the clinical trial source document, wherein generating the certified electronic copy comprises:
associating an electronic signature data element representing a signature of a user of the electronic clinical trial protocol management system with the electronic copy, the electronic signature resulting from an action by the user in association with a verification of the electronic copy, and
generating a data structure, in the memory of the electronic clinical trial protocol management system, linking the electronic signature data element with the electronic copy of the clinical trial source document.

33. The machine readable hardware storage medium of claim 32, wherein the method further comprises:
displaying, via the first portion of the GUI, at least a portion of the certified electronic copy of the clinical trial source document.

34. The machine readable hardware storage medium of claim 32, wherein the verification is related to a regulatory requirement.

35. The machine readable hardware storage medium of claim 34, wherein generating the certified electronic copy further comprises:
associating a certification statement related to the regulatory requirement with the electronic signature data element and/or the electronic copy of the clinical trial source document.

36. The machine readable hardware storage medium of claim 32, wherein the method further comprises:
deconstructing the electronic copy of the clinical trial source document into one or more displayable pages; and
associating the electronic signature data element representing the signature of the user of the electronic clinical trial protocol management system with each page of the one or more displayable pages.
